(19) United States
(12) Patent Application Publication
(10) Pub. No.: US 2012/0208767 A1
(43) Pub. Date: Aug. 16, 2012

(54) Method to Detect or Treat Painful Bladder Syndrome based on Relation to Quinone Oxidoreductase 1

(75) Inventor: Anil K. Jaiswal, Clarksville, MD (US)

(73) Assignee: University of Maryland, Baltimore, Baltimore, MD (US)

(21) Appl. No.: 13/372,634

(22) Filed: Feb. 14, 2012

Related U.S. Application Data
(60) Provisional application No. 61/442,422, filed on Feb. 14, 2011.

(51) Int. Cl. A61K 31/26; A61K 31/05; A61P 39/06; A61K 31/085; A61K 38/02; A61P 13/10; C12Q 1/68; A61K 31/357

(52) U.S. Cl. 514/18.9; 435/6.11; 514/514; 514/733; 514/452; 514/718; 514/731; 514/21.92

(57) ABSTRACT

A method includes obtaining a biological sample from a subject that does not display a symptom of Painful Bladder Syndrome (PBS). The method also includes analyzing the biological sample for a mutated form of NAD(P)H dehydrogenase [quinone], also known as Quinone Oxidoreductase 1, abbreviated NQO1, or a mutation of a NQO1 gene. The method also includes determining a risk of PBS for the subject based on analyzing the biological sample. In some methods, the mutation determined to present an increased risk is a cytosine to thymine nucleotide substitution at a position that leads to a proline to serine amino acid substitution at amino acid position 187 to produce a protein NQO1P187S. Another method includes selecting a subject that has above normal risk of Painful Bladder Syndrome (PBS) or is expressing a symptom of PBS, and treating the subject with a therapeutically effective amount of an antioxidant.

### CROSS-REFERENCE TO RELATED APPLICATIONS

This application claims benefit of Provisional Appln. 61/442,422, filed Feb. 14, 2011, the entire contents of which are hereby incorporated by reference as if fully set forth herein, under 35 U.S.C. §119(e).

### STATEMENT OF GOVERNMENTAL INTEREST

This invention was made with Government support under Grant No. ES007943 awarded by the National Institutes of Health. The Government has certain rights in the invention.

### BACKGROUND OF THE INVENTION

Painful Bladder Syndrome (PBS), also known as interstitial cystitis (IC), is a painful disease usually associated with advanced age and it primarily affects women more than men. The pathology of PBS develops sequentially with the formation of lesions on the interior of the bladder wall, which is lined with a layer of cuboidal luminal epithelium. This is followed by an eventual degradation of the contact between the luminal epithelium and the basal myoepithelial layers which form the bulk of the bladder wall. This loss of contact leads to intense pain and impairment of bladder function, for which effective diagnostic tools and treatment has not been developed. The molecular causes of PBS have yet to be elucidated, although there is increasing evidence which suggests the centrality of damage to luminal epithelial cell layer in the development of this disease.

There are several key features of PBS that are relevant for investigating the pathophysiological features including, but not limited to, alterations in genes regulating apoptosis and cell proliferation (e.g., see Keay S., “Cell signaling in interstitial cystitis/painful bladder syndrome,” Cell Signal, v20, no. 12, pp 2174-2179, 2008), defects in luminal epithelium (e.g., see Hurst, R. E., R. M. Moldwin, S. G. Mulholland, “Bladder defense molecules, urothelial differentiation, urinary biomarkers, and interstitial cystitis,” Urology; v69, no. 4 Suppl., pp S17-S23, 2007), mast cell invasion (e.g., see Sant G. R, D. Kempuraj, J. E. Marchand, et al. “The mast cell in interstitial cystitis: role in pathophysiology and pathogenesis,” Urology; v69, no. 4 Suppl., pp 34-40, 2007), and possibly autoimmune involvement (e.g., see: Stone, A. R., K. B. Quattrocchi, C. H. Miller, et al., “Role of the immune system in interstitial cystitis,” Semin Urol, v9, pp 108-114, 1991; and, Ratliff T., C. G. Klutke, M. Hofineister, et al., “Role of the immune response in interstitial cystitis,” Clin Immunol Immunopathol, v74, pp 209-216, 1995). However, an animal model of PBS had not been developed and genetic biomarker(s) of susceptibility to PBS remained unknown.

### SUMMARY OF THE INVENTION

Applicants have developed an animal model for Painful Bladder Syndrome (PBS) and identified a genetic marker and corresponding protein in the animal model and confirmed the genetic marker in humans. This development supports methods for detection or treatment of PBS, or both, based on the genetic marker or the corresponding protein and their roles in various signaling pathways.

In a first set of embodiments, a method includes obtaining a biological sample from a subject that does not display a symptom of Painful Bladder Syndrome (PBS). The method also includes analyzing the biological sample for a mutated form of NAD(P)H dehydrogenase [quinone], also known as Quinone Oxidoreductase 1, abbreviated NQO1, or a mutation of a NQO1 gene. The method also includes determining a risk of PBS for the subject based on analyzing the biological sample.

In some embodiments of the first set, determining the risk of PBS also includes determining that the subject has an above normal risk of developing PBS if the mutation in the NQO1 gene is detected in a DNA sample.

In some embodiments of the first set, the mutation is a cytosine to thymine nucleotide substitution at a position that leads to a proline to serine amino acid substitution at amino acid position 187 to produce a protein NQO1P187S.

In some embodiments of the first set, determining the risk of PBS includes determining that the subject has an above normal risk of developing PBS if the protein NQO1P187S is detected in the biological sample.

In some embodiments of the first set, determining that the subject has an above normal risk includes determining that the subject is heterozygous in the mutated NQO1 gene.

In some embodiments of the first set, determining that the subject has an above normal risk includes determining that the subject is homozygous in the mutated NQO1 gene.

In a second set of embodiments, a method includes selecting a subject that has above normal risk of Painful Bladder Syndrome (PBS) or is expressing a symptom of PBS, and treating the subject with a therapeutically effective amount of an antioxidant.

In some embodiments of the second set, the method includes obtaining a biological sample from the subject, and analyzing the biological sample to determine whether the subject is homozygous in a mutated NQO1 gene. This method includes administering a different second treatment if the subject is determined to be homozygous in the mutated NQO1 gene, and administering a different third treatment if the subject is determined to be not homozygous in the mutated NQO1 gene.

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a flow chart that illustrates at a high level an example method for detecting risk for Painful Bladder Syndrome (PBS) or treating PBS or both, according to an embodiment;

FIG. 2A through FIG. 2E are micrographs or graphs that illustrate example progression of PBS in a mouse model for PBS (homozygous in mutated NQO1 gene, signified by NQO1−/−) compared to wild-type (WT) mouse, according to an embodiment; FIG. 2E is a table that illustrates example incidence of PBS in wild type, heterozygous (designated NQO1+/−) and homozygous (NQO1−/−) genotypes for mutated NQO1 gene by age and gender.

FIG. 3A through FIG. 3E are micrographs or graphs that illustrate example apoptotic signaling in a mouse model for PBS compared to wild-type mouse, according to an embodiment;

FIG. 4A through FIG. 4C are graphs that illustrate example modulation of signaling cascades in a mouse model for PBS compared to wild-type mouse, according to an embodiment;

FIG. 5A through FIG. 5D are micrographs or graphs that illustrate example mitochondrial aberrations in a mouse model for PBS compared to wild-type mouse, according to an embodiment;

FIG. 6A through FIG. 6E are Western blot images that illustrate example NQO1 regulation of energy metabolic regulator factor PGC1α in a bladder of a mouse model for PBS compared to wild-type mouse, according to an embodiment

FIG. 7A and FIG. 7B are flow charts that illustrates model pathways of PBS pathology in a mouse model for PBS, according to an embodiment;

FIG. 8 is a photograph that illustrates example genotyping of normal human and human PBS patients; according to an embodiment.

### DETAILED DESCRIPTION

One or more methods are described for detecting or treating Painful Bladder Syndrome (PBS). In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention. Various references are cited in the following. The entire contents of the following references are hereby incorporated by reference as if fully set forth herein, except for terminology that is inconsistent with the terminology used herein.

Some embodiments of the invention are described below in the context of a mouse model that has PBS. However, the invention is not limited to this context. In other embodiments the subject is a human being or other animal who might be at risk for PBS or express symptoms of PBS or might not be at risk for PBS or not express symptoms of PBS.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The following terms as used herein have the corresponding meanings given here.

DEFINITIONS TABLE

| | |
|---|---|
| allele | a particular form of a genetic locus, distinguished from other forms by |
| | its particular nucleotide sequence, or one of the alternative |
| | polymorphisms found at a polymorphic site. |
| allele frequency | a measure of the relative frequency of an allele at a genetic locus in a |
| | population. Usually allele frequency is expressed as a proportion or a |
| | percentage. In population genetics, allele frequencies are used to depict |
| | the amount of genetic diversity at the individual, population, or species |
| | level. There are various databases in the public domain that contain |
| | SNPs and a user may for example, determine the relative allele |
| | frequency in some instances using such publicly available databases. |
| amino acid | A molecule containing an amine group, a carboxylic acid group, and a |
| | side-chain that is specific to each amino acid. The key elements of an |
| | amino acid are carbon, hydrogen, oxygen, and nitrogen. Chains of |
| | amino acids form proteins or portions thereof. |
| array | an arrangement of molecules, such as biological macromolecules (such |
| | as polypeptides or nucleic acids) or biological samples (such as tissue |
| | sections), in addressable locations on or in a substrate. A “microarray” |
| | is an array that is miniaturized so as to require or be aided by |
| | microscopic examination for evaluation or analysis. Arrays are |
| | sometimes called DNA chips or biochips. |
| effective amount | an amount of a therapeutic agent, which produces a desired effect |
| gene | a segment of DNA involved in producing a polypeptide chain. |
| | Specifically, a gene includes, without limitation, regions preceding and |
| | following the coding region, such as the promoter and 3′-untranslated |
| | region, respectively, as well as intervening sequences (introns) between |
| | individual coding segments (exons) |
| Genetic | susceptibility of a subject to a disease, such as PBS. Detecting a genetic |
| predisposition | predisposition can include, but does not necessarily include, detecting |
| | the presence of the disease itself, such as but not limited to an early |
| | stage of the disease process. Detecting a genetic predisposition also |
| | includes detecting the risk of developing the disease, and determining |
| | the susceptibility of that subject to developing the disease or to having |
| | a poor prognosis for the disease. Thus, if a subject has a genetic |
| | predisposition to a disease they do not necessarily develop the disease |
| | but are at a higher than normal risk for developing the disease. |
| Genomic target | A sequence of nucleotides located in a particular region in the human |
| sequence | genome that corresponds to one or more specific genetic abnormalities, |
| | such as a nucleotide polymorphism, a deletion, an insertion, or |
| | amplification. The target can be for instance a coding sequence; it can |
| | also be the non-coding strand that corresponds to a coding sequence. |
| | The target can also be a non-coding sequence, such as an intronic |
| | sequence |
| genotype | an unphased 5′ to 3′ sequence of nucleotide pair(s) found at a set of one |
| | or more polymorphic sites in a locus on a pair of homologous |
| | chromosomes in an individual. |
| haplotype | a 5′ to 3′ sequence of nucleotides found at a set of one or more |
| | polymorphic sites in a locus on a single chromosome from a single |
| | individual. “Haplotype pair” is the two haplotypes found for a locus in |
| | a single individual. |
| heterozygous | an organism in which the two alleles of a gene code for different |
| | proteins |
| homozygous | an organism in which both alleles of a gene code for the same protein |
| missense | A mutation that introduces a different amino acid into the sequence of |
| mutations | the encoded protein. |
| mutation | Any change of a nucleic acid sequence as a source of genetic variation. |
| | For example, mutations can occur within a gene or chromosome, |
| | including specific changes in non-coding regions of a chromosome, for |
| | instance changes in or near regulatory regions of genes. Types of |
| | mutations include, but are not limited to, base substitution point |
| | mutations (which are either transitions or transversions), deletions, and |
| | insertions. |
| NQO1 | NAD(P)H dehydrogenase [quinone], also called Quinone |
| | Oxidoreductase 1, a protein that serves as cytosolic phase II |
| | biotransformation enzyme whose primary physiological role is catalysis |
| | of two-electron reduction of quinones and thereby their detoxification. |
| NQO1 gene | a gene in a position that normally codes for NQO1 |
| NQO1P187S | a mutated form of NQO1 formed by a proline to serine amino acid |
| | substitution at amino acid position 187. The NQO1P187S mutant |
| | protein is rapidly degraded by proteasomes. |
| nucleic acid | a molecule comprising a sequence of one or more repeating chemical |
| | units known as “nucleotides” or “bases.” There are four bases in |
| | deoxyribonucleic acid (DNA): adenine, thymine, cytosine, and guanine, |
| | represented by the letters A, T, C and G, respectively. In Ribonucleic |
| | acid (RNA) the base uracil (U) replaces the base thymine (T). |
| PCR | a polymerase chain reaction technique used to amplify a single or few |
| | copies of a portion of DNA across several orders of magnitude, |
| | generating thousands to millions of copies of a particular DNA |
| | sequence |
| PBS | Painful Bladder Syndrome, also called interstitial cystitis (IC). |
| polymorphism | A variation in a sequence of a gene. A polymorphisms can be a |
| | variation (DNA sequence difference) which produce functionally |
| | equivalent gene products; and can also refer to variants in the DNA |
| | sequence which can lead to gene products that are not functionally |
| | equivalent. |
| prophylactically | an amount of a therapeutic agent, which, when administered to a |
| effective amount | subject, will have the intended prophylactic effect, e.g., preventing or |
| | delaying the onset (or reoccurrence) of a disease or set of one or more |
| | symptoms, or reducing the likelihood of the onset (or reoccurrence) of |
| | the disease or set of symptoms. The full prophylactic effect does not |
| | necessarily occur by administration of one dose and may occur only |
| | after administration of a series of doses. Thus, a prophylactically |
| | effective amount may be administered in one or more administrations |
| protein | a generic term referring to native protein, fragments, peptides, or |
| | analogs of a polypeptide sequence. Synonym used herein includes |
| | “polypeptide.” Hence, native protein, fragments, and analogs are |
| | species of a polypeptide genus |
| reference allele, | a genotype that predominates in a natural population of organisms that |
| reference gene | do not have a disease process, such as PBS. The reference genotype |
| | differs from mutant forms. |
| risk allele, | an allele (or gene) associated with a particular type or form of disease. |
| risk gene | The risk allele identifies a tag single nucleotide polymorphism that can |
| | be used to detect or determine the risk for a disease, such as PBS. |
| sample | includes any biological specimen obtained from a subject |
| SNP | a single nucleotide polymorphism. A single base (nucleotide) |
| | polymorphism in a DNA sequence among individuals in a population. |
| | SNPs within a coding sequence will not necessarily change the amino |
| | acid sequence of the protein that is produced, due to degeneracy of the |
| | genetic code. A SNP in which both forms lead to the same polypeptide |
| | sequence is termed “synonymous” (sometimes called a silent |
| | mutation)--if a different polypeptide sequence is produced they are |
| | “nonsynonymous”. A nonsynonymous change may either be missense |
| | or “nonsense”, where a missense change results in a different amino |
| | acid, while a nonsense change results in a premature stop codon. |
| subject | An organism that is an object of a method or material, including |
| | mammals, e.g., humans, dogs, cows, horses, kangaroos, pigs, sheep, |
| | goats, cats, mice, rabbits, rats, and transgenic non-human animals. |
| | Synonyms used herein include “patient” and “animal” |
| tag SNP | a SNP that by itself or in combination with additional tag SNPs |
| | indicates the presence of a specific haplotype, or of one member of a |
| | group of haplotypes. The haplotype or haplotypes can indicate a genetic |
| | factor associated with risk for disease, thus a tag SNP or combination |
| | of tag SNPs indicates the presence or absence of risk factors for |
| | disease. |
| therapeutic agent | an inhibitor or enhancer of one or more enzymes in a targeted pathway |
| therapeutically | an amount of a therapeutic agent, which achieves an intended |
| effective amount | therapeutic effect in a subject., e.g., eliminating or reducing the severity |
| | of a disease or set of one or more symptoms The full therapeutic effect |
| | does not necessarily occur by administration of one dose and may occur |
| | only after administration of a series of doses. Thus, a therapeutically |
| | effective amount may be administered in one or more administrations |
| treating | taking steps to obtain beneficial or desired results, including clinical |
| | results, such as alleviating or ameliorating one or more symptoms of a |
| | disease; diminishing the extent of disease; delaying or slowing disease |
| | progression; ameliorating and palliating or stabilizing a metric |
| | (statistic) of disease. “Treatment” refers to the steps taken. |

It is to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below

### 1. Overview

Applicant has determined that a lack of Quinone Oxidoreductase 1 (NQO1) and the gene that codes for it play an important role in risk for and development of Painful Bladder Syndrome (PBS). In particular, the presence of a mutated NQO1 gene that codes for the mutated protein NQO1P187S form of NQO1 instead of NQO1 plays a major role so that either the mutated NQO1 gene or the mutated form of NQO1 serves as a biomarker for PBS. Details of these risks and the effect of NQO1 on cascading pathways during development of PBS are described in a mouse model embodiment, described in more detail in a later section. Treatment is proposed to mitigate the effects at one or more points of the cascading pathways.

NQO1 is a cytosolic phase II biotransformation enzyme whose primary physiological role is catalysis of two-electron reduction of quinones and thereby their detoxification (e.g., see Ross D, J. K. Kepa, S. L. Winski, et al., “NAD(P)H:quinone oxidoreductase 1 (NQO1): chemoprotection, bioactivation, gene regulation and genetic polymorphism,” Chem Biol Interact v129, pp 77-97, 2000). NQO1 is measurably expressed in every tissue including bladder and its expression is induced downstream of exposure to radiation and chemical stress (e.g., see Ross [2000]; Park S, H. Zhao, M. R. Spitz, et al., “An association between NQO1 genetic polymorphism and risk of bladder cancer,” Mut Res v536, pp 131-137, 2003; and Kaspar J, S. K. Niture, A. K. Jaiswal. “Nrf2:INrf2 (Keap1) signaling in oxidative stress,” Free Rad Biol Med v47, pp 1304-1309, 2009). Epidemiological studies have shown a correlation between NQO1P187S mutation leading to loss of enzyme with bladder cancers in smokers (e.g., see Park [2003]; and Sanyal S, C. Ryk, P. J. De Verdier, et al., “Polymorphisms in NQO1 and the clinical course of urinary bladder neoplasms,” Scand J Urol Nephrol, v41, pp 182-190, 2007).

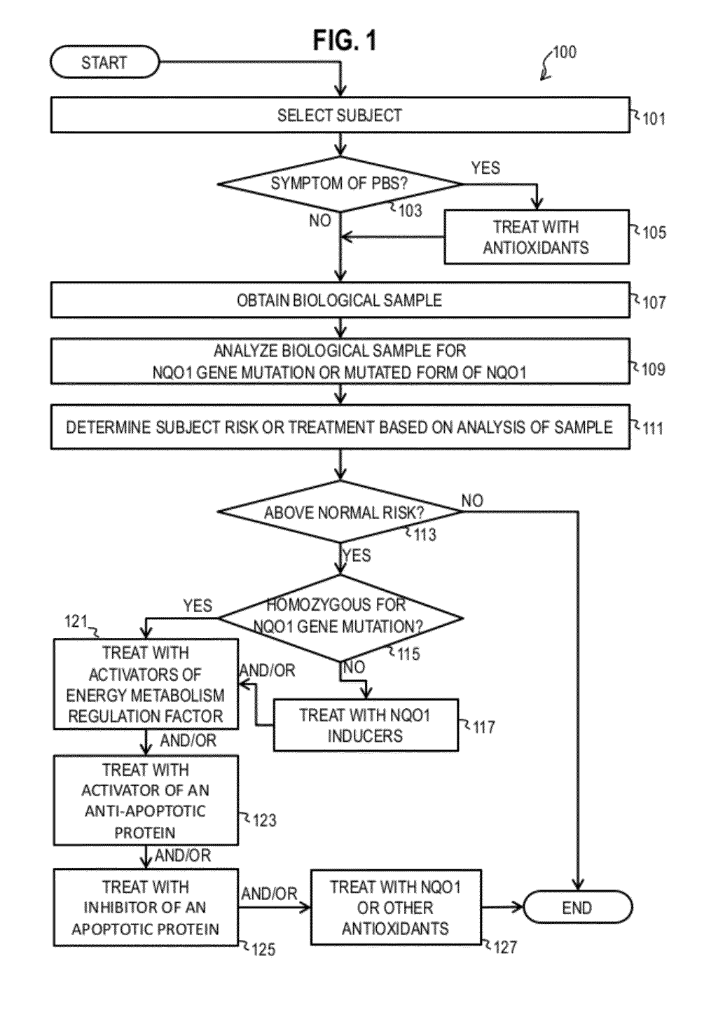

FIG. 1 is a flow chart that illustrates at a high level an example method 100 for detecting risk for Painful Bladder Syndrome (PBS) or treating PBS or both, according to an embodiment. Although steps are depicted in FIG. 1, as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 101, a subject is selected, such as a wild type mouse, a mouse model for PBS, a human being expressing no symptoms of PBS, a human being expressing no symptoms of PBS but considered at elevated risk of PBS, such as a woman of advanced age, or a human being expressing one or more symptoms of PBS.

In step 103, it is determined whether the subject already expresses one or more symptoms of PBS. If so, then in step 105 the subject is treated by administering a therapeutically effective amount of one or more antioxidants. The rationale for this treatment is that NQO1 possesses the enzymatic activity of two-electron reduction of quinones within the cytosol, which classifies NQO1 as an antioxidant enzyme. This antioxidant activity serves to protect bladder epithelial cells against accumulation of high levels of reactive oxygen species (ROS), as is evidenced by such accumulation after environmental chemical insult. Insofar as it is this protection against redox imbalance afforded by constitutive expression of NQO1 in bladder epithelium which preserves this layer from the progression of PBS in adult mice, antioxidant activity by other agents is anticipated to serve to mitigate the lack of NQO1 in subjects suffering from PBS.

Whether the subject expresses one or more symptoms of PBS or not, in the illustrated embodiment, a biological sample is obtained in step 107. Biological samples include all clinical samples useful for detection of PBS in subjects, such as cells, tissues, and bodily fluids, for example blood and saliva; derivatives and fractions of blood, such as serum; extracted galls; biopsied or surgically removed tissue, including tissues that are, for example, unfixed, frozen, fixed in formalin and/or embedded in paraffin; tears; milk; skin scrapes; surface washings; urine; sputum; cerebrospinal fluid; prostate fluid; pus; or bone marrow aspirates. In a particular example, a sample includes blood obtained from a human subject, such as whole blood or serum. In another particular example, a sample includes buccal cells, for example collected using a swab or by an oral rinse. In some embodiments, step 107 includes obtaining DNA sequence data previously obtained from the subject.

In step 109 the biological sample is analyzed to detect a mutation in the NQO1 gene or a mutated form of NQO1 that does not provide the protection from PBS afforded by NQO1 and the reference NQO1 gene. For example, in some embodiments, a DNA sample is analyzed to detect a single nucleotide polymorphism that involves a cytosine to thymine nucleotide substitution at a position in the DNA seqeunce that leads to a proline to serine amino acid substitution at amino acid position 187 to produce the protein NQO1P187S. In some embodiments, a biological sample is analyzed to detect the protein NQO1P187S, itself.

Any experimental method for analyzing a DNA sample may be used, as described later in the experimental methods section. In an example embodiment, DNA at the site of the NQO1 gene was amplified by polymerase chain reaction (PCR), digested with DNA restriction enzyme Hinf I and separated on agarose gel. DNA of the reference NQO1 gene is undigested and moves a shorter distance through the gel. The SNP mutation described in the previous paragraph creates a DNA restriction enzyme Hinf I site and DNA of the mutated NQO1 gene is digested. The digested fragments of the mutated NQO1 gene move farther through the gel and are thus separated from the undigested reference NQO 1 gene

In step 111, risk or treatment is determined based on the analysis of the biological sample. For example, in some embodiments, it is determined that the subject has an above normal risk of developing PBS if the mutation in the NQO1 gene is detected in the DNA sample. This is based on the data described below with reference to experimental embodiments on the mouse PBS model and with reference to the experimental embodiment on DNA samples from human PBS patients. In some embodiments, it is determined that the subject has an above normal risk of developing PBS if the mutated form of NQO1, such as NQO1P187S, was detected in the biological sample in step 109.

Immunoblot/Western analysis with anti-NQO1 antibody may be used to predict normal NQO1 or mutant NQO1P187S. This assay is based on the fact that NQO1P187S mutant protein is unstable and degraded by proteasomes. Therefore, partial or complete absence of NQO1 protein will suggest NQO1P187S mutant protein.

In some embodiments, step 111 includes determining whether the subject is heterozygous in the mutated NQO1 gene or homozygous in the mutated NQO1 gene. Both homozygous and heterozygous NQO1 individuals are susceptible and at above average risk for PBS, as demonstrated in the human embodiment, described in a later section. For example, in some embodiments, step 111 includes determining that the subject is heterozygous in mutated NQO1 gene if the mutation in the NQO1 gene is detected in the DNA sample and the reference NQO1 gene is also detected in the DNA sample. In some embodiments, step 111 includes determining that the subject is heterozygous in mutated NQO1 gene if the mutated form of NQO1 is detected in the biological sample and NQO1 is also detected in the biological sample.

As a further example, in some embodiments, step 111 includes determining that the subject is homozygous in the mutated NQO1 gene if the mutation in the NQO1 gene is detected in the DNA sample and if the reference NQO1 gene is not also detected in the DNA sample. In some embodiments, step 111 includes determining that the subject is homozygous in the mutated NQO1 gene if the mutated form of NQO1 is detected in the biological sample and if NQO1 is not also detected in the biological sample.

In various embodiments, the determinations are made in the laboratory, or a kit is used to make the determination, or an array is used to make the determination, as described below in later sections. For example, in some embodiments, the determination is made using an array that includes probes for the mutated NQO1 gene and the reference NQO1 gene or with probes for NQO1 and NQO1P187S.

In some embodiments, the determinations made in step 111 suffice; and, the process ends with a risk assessment of normal risk or above normal risk for the subject. In such embodiments, the following steps are omitted. However, in some embodiments, treatment is determined based on the determinations made in step 111.

In step 113, it is determined whether the subject has above normal risk, e.g., because it was determined in step 111 that either the mutated NQO1 gene was detected or the mutated form of NQO1 was detected during step 109. If not, no treatment is indicated; and, the process ends.

If it is determined in step 113, that the subject has above normal risk, then in step 115 it is determined whether the subject is homozygous in the mutated NQO1 gene, e.g., because it was determined in step 111 that either the reference NQO1 gene was not detected or NQO1 was not detected during step 109.

If the subject is not homozygous in the mutated NQO1 gene, then one allele codes for NQO1; and, control passes to step 117 to treat with agents that induce NQO1 production, when treatment is indicated. Such agents are known in the art. The timing when treatment is indicated can be discovered through normal experimentation. It is anticipated that the timing for such treatment is when the subject is or is expected to undergo stress, such as when exposed to trauma or surgery or toxins or advanced age or some combination.

Known inducers of NQO1 activity include Sulphoraphane from broccoli, antioxidants including 3-(2)-tert-butyl-4-hydroxyanisole (BHA) and Tertiary-butyl hydroquinone (t-BHQ), and Flavonoids including Silibinin and Honoki. 1-Isothiocyanato-4-methylsulfinylbutane (CAS No. 4478-93-7) is vailable from SIGMA ALDRICH™ of St. Louis, Mo. (Catalog No. 54441, >90%). 2-tert-Butyl-4-hydroxyanisole and 3-tert-butyl-4-hydroxyanisole, mixture (CAS No. 25013-16-5) is available from SIGMA ALDRICH™ (Cat. No. B1253). 2-(1,1-Dimethylethyl)-1,4-benzenediol (CAS No. 1948-33-0) is available from SIGMA ALDRICH™ (Cat. No. 112941). Silibinin is (2R,3R)-3,5,7-trihydroxy-2-[(2R,3R)-3-(4-hydroxy-3-methoxyphenyl)-2-(hydroxymethyl)-2,3-dihydrobenzo[b][1,4]dioxin-6-yl]chroman-4-one (CAS No. 22888-70-6) and is available from SIGMA ALDRICH™ (Cat. No. S0417). Honokiol is 2-(4-hydroxy-3-prop-2-enyl-phenyl)-4-prop-2-enyl-phenol (CAS No. 35354-74-6) and is available from SIGMA ALDRICH™ (Cat. No. H4914). A therapeutically effective amount, or a prophylactically effective amount, of one or more of these NQO1 inducers is administered during step 117.

If the subject is homozygous in the mutated NQO1 gene, then neither allele codes for NQO1 and control passes to step 121 or following steps to treat with one or more agents that mitigate the loss of NQO1. As described above, the timing when treatment is indicated can be discovered through normal experimentation. It is anticipated that the timing for such treatment is when the subject is or is expected to undergo stress, such as when exposed to trauma or surgery or toxins or advanced age or some combination.

In some embodiments, the method includes step 121, during which the subject is treated with one or more activators of energy metabolism regulation factor, for example to mitigate the structural damage to mitochondria described below with reference to the mouse model for PBS. An example activator is Peroxisome Proliferator Activated Receptor Gamma, Coactivator 1 alpha (also known as PPARGC1A or PGC1alpha), which is coded by Gene Accession Number NG_{—}028250. A therapeutically effective amount, or a prophylactically effective amount, of one or more of these activators is administered during step 121.

In some embodiments, the method includes step 123, during which the subject is treated with one or more activators of one or more anti-apoptotic proteins. Example activators include Bcl-xL, which is coded by Gene Accession Number NG_{—}029002, and Bcl-2, which is coded by Gene Accession number NG_{—}009361). A therapeutically effective amount, or a prophylactically effective amount, of one or more of these activators is administered during step 123.

In some embodiments, the method includes step 125, during which the subject is treated with one or more inhibitors of one or more apoptotic proteins. Example inhibitors include Bad (Protein Accession Number NP_{—}116784) and Bid (Protein Accession Number NP_{—}001231501). A therapeutically effective amount, or a prophylactically effective amount, of one or more of these inhibitors is administered during step 125.

In some embodiments, the method includes step 127, during which the subject is treated with NQO1 or one or more other antioxidants, or some combination. A therapeutically effective amount, or a prophylactically effective amount, of one or more of these antioxidants is administered during step 127.

Thus, method 100 provides for detection or treatment of PBS, or both, based on a genetic marker or the corresponding protein.

### 2. Example Embodiments

### 2.1 Mouse Model Embodiment

### 2.1.1 Materials and Protocols

Mice. C57/BL6 mice containing or lacking NQO1 were characterized previously (e.g., see Radjendirane V, P. Joseph, Y. H. Lee, et al., “Disruption of the DT diaphorase (NQO1) gene in mice leads to increased menadione toxicity,” J Biol Chem v273, pp 7382-738, 1998) and housed in University of Maryland Animal Housing facility using chow and water ad libitum. Female and male mice were allowed to remain until reaching appropriate age and then sacrificed according to IACUC-approved protocols. The target NQO1 gene is known to be in region 3 of chromosome 8 (8D3) of the mouse.

Morphological examination of bladders. Whole bladders from age-matched mice of both sexes, ages 6, 12, and 24 weeks, wild-type (WT), and homozygous in mutated NQO1 gene that codes for NQO1P187S (designated NQO1−/−), and heterozygous in mutated NQO1 gene that codes for NQO1P187S (designated NQO1+/−) were collected from isofluorane-euthanized mice and examined under both microscope-aided and unaided techniques. Examination focused on incidence of PBS symptoms and bladders observed to have features of PBS were noted as PBS-positive. In all groups, the number of samples (n)>10 mice.

Histochemistry and Immunohistochemistry. Whole bladders from age-matched mice were immediately fixed in 10% formalin at room temperature overnight and embedded in paraffin at University of Maryland Dermatopathology Laboratory (MDL). Sections were cut and mounted to charged slides, and Hematoxylin/Eosin stained slides were prepared at the MDL. Histochemistry slides were prepared: TUNEL assay performed on paraffin-embedded tissue sections, and trichrome staining performed. Immunohistochemistry was performed on remaining slides, using antibodies against Tryptase, p53, NFkB, PCNA, and ICAM.

Cell culture and Western blots. Cells were cultured in appropriate media (DMEM+1% p/s+10% FBS) and subcultured as necessary. Tissue homogenates were prepared by removing whole bladders from freshly-sacrificed mice killed with isoflurane inhalation and lysing tissue as a 10% homogenate in ice-cold RIPA buffer (50 mM Tris-Cl, pH 7.4, 150 mM NaCl, 1% NP-40, 1 mM EDTA, 0.5% Na Deoxycholate, and 1% Triton X100 with 1 mM PMSF and 1× Protease Inhibitor Cocktail (Complete, Roche)) using glass and Teflon pestle system, and further clarified via centrifugation at 14000 rpm at 4 degrees for 10 minutes. Homogenates were stored at −80° C. until use. Western blotting was performed essentially according to Towbin H, T. Staehelin, J. Gordon, “Electrophoretic transfer of proteins from polyacrylamide gels to nitrocellulose sheets: procedure and some applications,” Proc Natl Acad Sci USA, v76, pp 4350-4354, 1979.

Immunoprecipitation. Human bladder carcinoma T24 cell line was cultured in DMEM+10% FBS+1% p/s at 37 degrees in 5% carbon dioxide before IP studies. Cells were incubated with chemicals as necessary and washed 3× in PBS on ice. Cells were scraped into microfuge tubes using RIPA buffer and protein A/G beads (SANTA CRUZ BIOTECH™ of Santa Cruz, Calif.) were equilibrated with 1 mg aliquots for 1 h. After equilibration, beads were removed and lysates were incubated o/n with 1:100 of relevant antibodies, and next day incubated with 30 microliters A/G beads for 1 h before 3× RIPA buffer wash and boiling 5 mins.

Gene microarray studies. Microarray studies were performed using freshly-excised bladder tissue from age-matched 24 week old female WT and NQO1−/− mice. Bladders were stored until sufficient numbers were attained and groups of 3 were homogenized in glass pestles on ice to form a pool of total RNA using the RNEasy kit (QIAGEN™ of Hilden, Germany) and QiaShredder (QIAGEN™). Quality of purified RNA was verified using ABI Bioanalyzer (AGILENT TECHNOLOGIES™ of Wilmington, Del.) at the University of Maryland Microarray core facility, and pools meeting quality criteria were selected for further microarray studies. Samples were annealed to Mouse Genome 430 2.0 ST array (AFFYMETRIX™ of Santa Clara, Calif.) with 3 chips used per sample group, according to manufacturer instructions. Statistical and pathways analysis of results of microarray studies were performed by Dr. Conover Talbot at Johns Hopkins University Institute of Genetic Medicine and by Brad Patrick, using INGENUITY™ software (of Redwood City, Calif.) to perform statistical analysis of results using Spotfire software (TIBCO™ of Somerville, Mass.). Results were grouped according to physiological process and fold-change to facilitate review.

Reverse-transcriptase PCR. Primers for RT-PCR of MUP1 gene were designed using NCBI database transcript sequence and forward and reverse primers were designed. NQO1 primers were used as genotype markers and GAPDH primers were used as loading control. Reactions were performed using Superscript One-step RT-PCR kit (INVITROGEN™ of Carlsbad, Calif.) as per manufacturer recommendation, and separated on 1% agarose gel, and photographed.

Ex-vivo cell culture and chemical treatment. Whole bladders from age-matched female wild-type and NQO1−/− 24 weeks-old mice were excised from euthanized mice and minced in a 10 cm dish with a razorblade. Pieces were suspended in microfuge tubes containing 500 microliters of 1% Collagen reconstituted in PBS and incubated at 37 degrees with 700 rpm shaking for 45 minutes to dissociate cells from tissue. Tubes were centrifuged at 500 g for 3 minutes to pellet cells, and resuspended in full cell culture media. Cells were plated in 8-well tissue culture chamber slides (e.g., from LAB-TEK™) with cells from one bladder equally divided into one 8-well slide, into 1 mL media total volume. After 2-4 days of growth with daily media changes, cells were treated with various concentrations of benzopyrene dissolved in acetone, or acetone control in fresh media for 18 h, followed by removal and addition of DCF (SIGMA ALDRICH™) at 5 micromolar for 1 h. DCF was then removed and full media added for 1 h, followed by immediate mounting of cover slips in VectaShield (VECTOR LABS™ of Burlingame, Calif.) and viewing under FITC-viewing filter to determine fluorescence of ROS-modified DCF within cells.

Electron Microscopy. Tunneling electron microscopy studies were performed on fixed whole bladder tissues at the University of Maryland Electron Microscopy Core Facility. Whole bladders were fixed in a solution of 4% Paraformaldehyde/2.5% Glutaraldehyde overnight at 4 degrees C. from freshly-sacrificed mice. Bladders were embedded in successive concentrations of epoxy resin until encased and then sections trimmed until appropriate orientation and size was achieved. Sections were embedded on grids and stained with Uranyl Acetate/Lead (ref) and photographs were taken at 4400× and 30000× magnifications, using samples of 24 week old age-matched female WT and NQO1−/− (samples from 2 separate animals from each genotype). Bars on photographs indicate size references.

Statistical Analysis. All experiments were performed on at least 3 replicates and data collected were statistically analyzed where noted using Student's T test.

### 2.1.2 Results

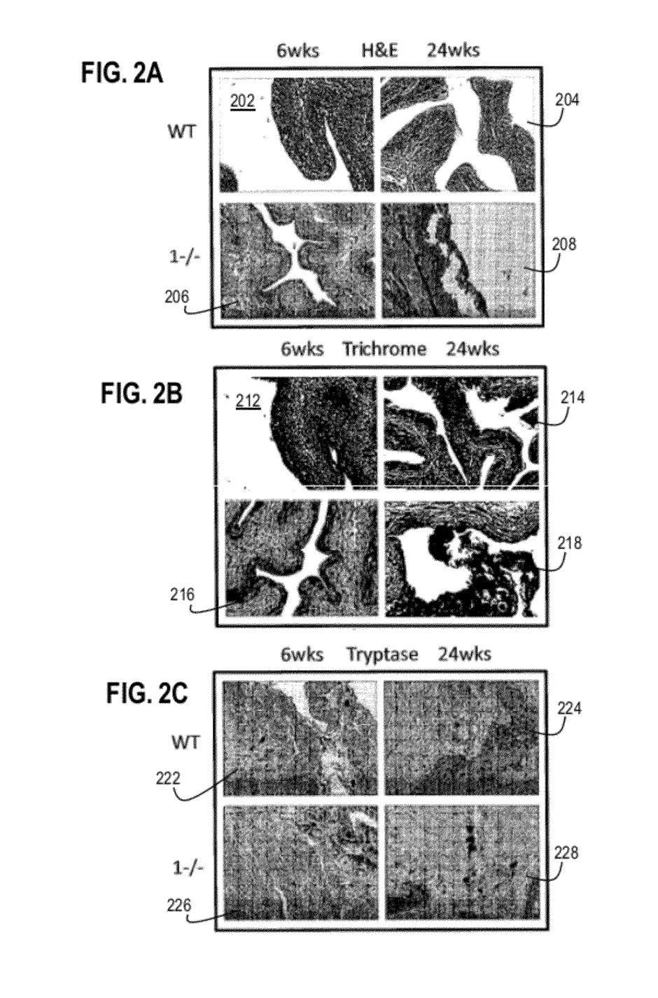

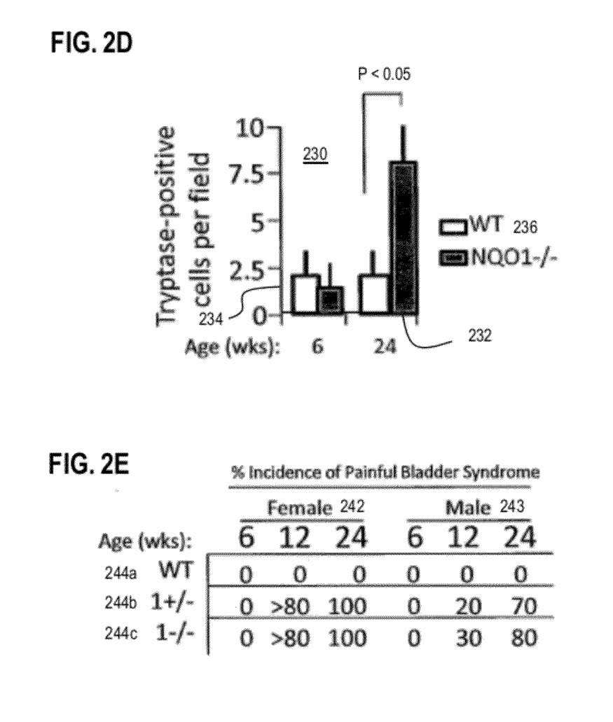

PBS dependence on NQO1 gene copy number. FIG. 2A through FIG. 2D are micrographs or graphs that illustrate example progression of PBS in a mouse model for PBS (homozygous in mutated NQO1 gene, signified by NQO1−/−) compared to wild-type (WT) mouse, according to an embodiment. FIG. 2A is a set of micrographs that illustrate example damage to luminal epithelium in NQO1−/− mice. FIG. 2A includes micrograph 202 of wild type mouse at 6 weeks, micrograph 204 of wild type mouse at 24 weeks, micrograph 206 of NQO1−/− mouse at 6 weeks, and micrograph 208 of NQO1−/− mouse at 24 weeks. Bladders from wild-type and NQO1−/− mice (both 6 and 24 week old, age-matched female) were fixed in formalin and embedded in paraffin, and sections were stained with hematoxylin & eosin. These micrographs illustrate that in wild-type bladders luminal epithelium was intact at both 6 and 24 weeks, while luminal epithelium in bladders from NQO1−/− mice had profound loss of integrity from 6 to 24 weeks of age.

Similarly, FIG. 2B is a set of micrographs that also illustrate example damage to luminal epithelium in NQO1−/− mice with Trichrome staining. FIG. 2B includes micrograph 212 of wild type mouse at 6 weeks, micrograph 214 of wild type mouse at 24 weeks, micrograph 216 of NQO1−/− mouse at 6 weeks, and micrograph 218 of NQO1−/− mouse at 24 weeks. These trichrome-stained tissue sections show a detachment of the luminal epithelium from the underlying basolateral layer in NQO1−/− mice at 24 weeks of age, which was not present at 6 weeks, or at any age in wild-type mice.

To investigate whether there was an immune response present in affected bladders tissue sections were stained for Tryptase, a well-characterized marker for mast cell invasion of tissues. FIG. 2C is a set of micrographs that illustrate example mast cell invasion in NQO1−/− mice with Tryptase staining. FIG. 2C includes micrograph 222 of wild type mouse at 6 weeks, micrograph 224 of wild type mouse at 24 weeks, micrograph 226 of NQO1−/− mouse at 6 weeks, and micrograph 228 of NQO1−/− mouse at 24 weeks. Paraffin-embedded sections (6 and 24 week old female) were stained with anti-Tryptase antibodies and stained with Vector Red with hematoxylin counterstain. Data from such micrographs plotted in a bar graph. FIG. 2D is a bar graph 230 that illustrates example increase in number of cells that stain positive to Tryptase, an indication of mast cell invasion. The horizontal axis 232 indicates age of mouse and the vertical axis 234 indicates the number of cells positive stained with Tryptase in a field of view. The open bars 236 indicate data for wild-type mice and the filled bars 238 indicate data for NQO1−/− mice averaged over 10 fields. Graph 230 shows a significant increase in mast cell invasion of bladder tissues in NQO1−/− bladders at 24 weeks of age, compared to wild-type or either mouse type at 6 weeks. Error bars are standard deviation of averaged data. The increased mast cell invasion is 95% likely to be non-random (P<0.05).

FIG. 2E is a table that illustrates example incidence of PBS in wild type, heterozygous (designated NQO1+/−) and homozygous (NQO1−/−) genotypes for mutated NQO1 gene by age and gender. Row 244a presents the data for wild type mice; row 244b presents the data for NQO1+/− mice; and, row 244c presents the data for NQO1−/− mice. The data for female mice is presented in columns 242 and for male mice in columns 243. In each set of columns, one column shows data for 6 weeks, the next for 12 weeks and the third for 24 weeks. Twenty mice in each group were harvested; bladders were dissected and examined visually and via microscopy to determine bladder health and incidence of PBS. Bladders showing symptoms consistent with PBS were counted as PBS-positive. In these morphological studies of whole bladders excised from 6-, 12- and 24-weeks old female and male mice of wild-type and NQO1−/− genotypes there was a profound increase of PBS observed in female NQO1−/− mice that were at least 12 weeks of age, which was not present in juvenile NQO1−/− mice (6 weeks old. Twelve-weeks and older male NQO1−/− mice also showed PBS, but at lower magnitude than female mice. PBS was not observed in any mice from the wild-type population. These results suggest that PBS is both age and sex driven, which is regulated by the presence of NQO1 in bladder. Further, this data shows that female and male heterozygous mice (NQO1+/−) at similar ages exhibited more or less similar morphology of PBS to homozygous (NQO1−/−) mice, suggesting that the reference NQO1 gene must be present in both alleles to prevent this pathology.

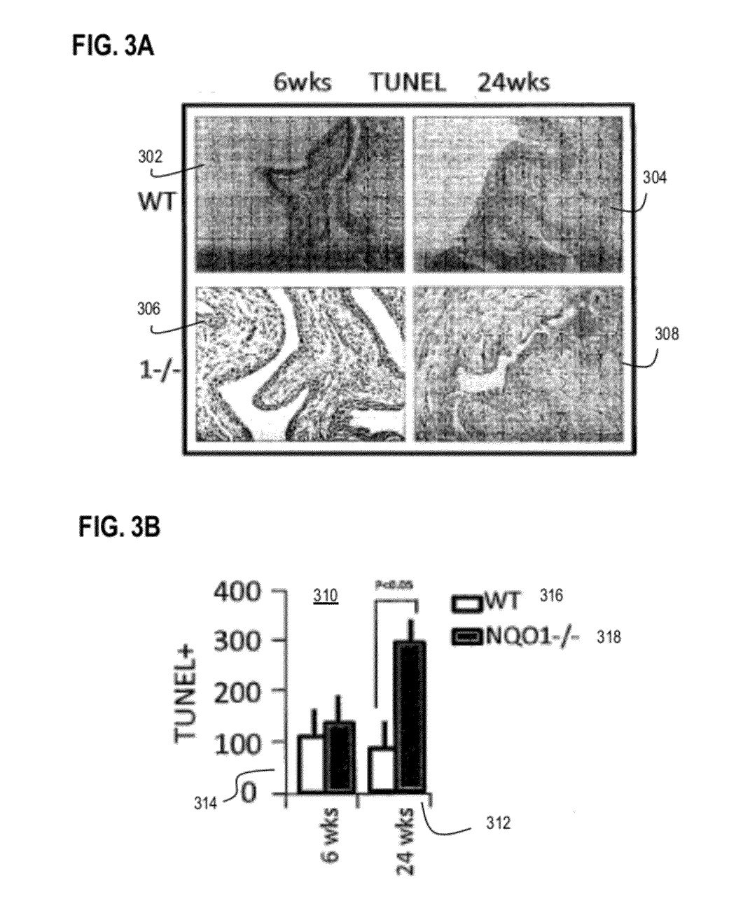

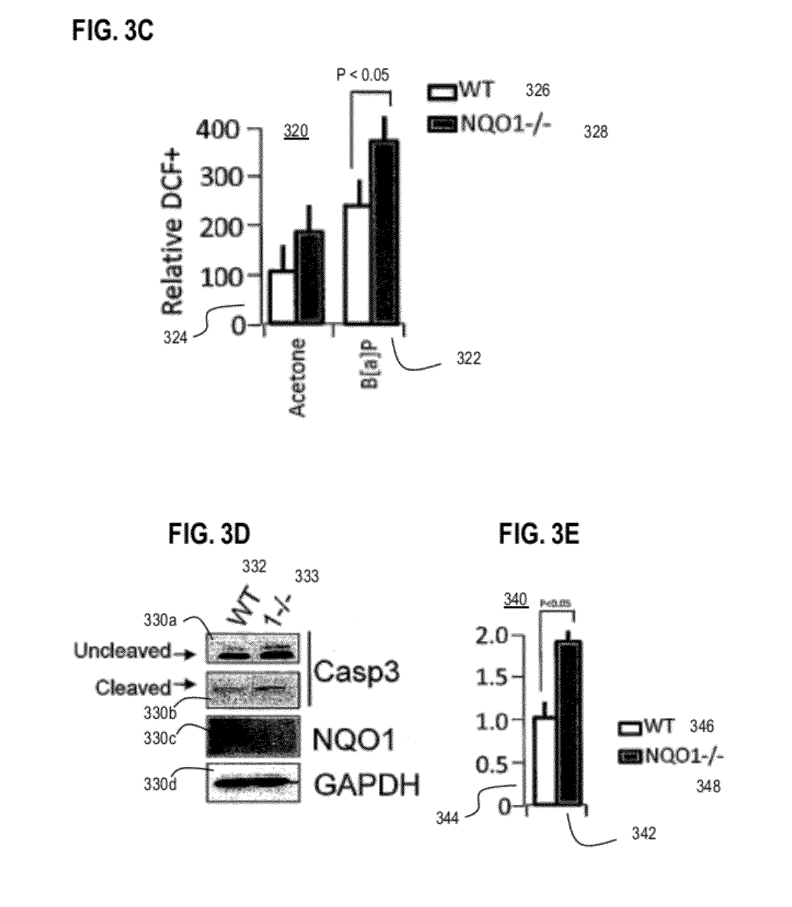

Increased apoptotic signaling in NQO1-null, PBS-positive bladders. The biological effects of the PBS morphology on bladder tissues was also investigated. Sections of bladder from 6- and 24-weeks old wild-type and NQO1−/− mice were stained for terminal deoxynucleotidyl transferase dUDP nick-end labeling (TUNEL), a well-characterized marker for DNA fragmentation in cells. FIG. 3A through FIG. 3E are micrographs or graphs that illustrate example apoptotic signaling in a mouse model for PBS compared to wild-type mouse, according to an embodiment. FIG. 3A is a set of micrographs that illustrate example damage to luminal epithelium in NQO1−/− mice. FIG. 2A includes micrograph 302 of wild type mouse at 6 weeks, micrograph 304 of wild type mouse at 24 weeks, micrograph 306 of NQO1−/− mouse at 6 weeks, and micrograph 308 of NQO1−/− mouse at 24 weeks. Paraffin-embedded sections from bladders (6 and 24 weeks old) were stained for terminal deoxynucleotidyl dUTP nick-end labeling (TUNEL) stain to detect fragmentation of genomic DNA. TUNEL-positive nuclei are stained dark. FIG. 3B is a bar graph 310 that illustrates example DNA fragmentation dependence on genotype and age. The horizontal axis 312 indicates age of mouse and the vertical axis 314 indicates the number of cells positive stained for TUNEL. The open bars 316 indicate data for wild-type mice and the filled bars 318 indicate data for NQO1−/− mice. Graph 310 shows that there was a slight increase in DNA damage at 6 weeks in NQO1−/− over wild-type bladder epithelium, but, that there was a much larger difference in TUNEL-positive cells along the luminal epithelium at 24 weeks.

Additionally, stressors have been shown to play a role in bladder pathology (e.g., see Robbins M. T., J. DeBerry, T. J. Ness, “Chronic physiological stress enhances nociceptive processing in the urinary bladder in high-anxiety rats,” Physiol Behav, v91, pp 544-550, 2007) and PBS patients may be more likely to accrue further environmental stress-related tissue damage due to weaker nociception (e.g., see Kilpatrick L. A., E. Ornitz, H. Ibrahimovic, et al., “Gating of sensory information differs in patients with interstitial cystitis/painful bladder syndrome,” J Urol, v184, no. 3, pp 958-963, 2010). Therefore, the role of environmental stressors on bladder stress signaling was investigated. In ex vivo studies where cells from wild-type and NQO1−/− bladders were cultured and subjected to environmental insult via the well-characterized hydrocarbon benzo[α]pyrene (B[α]P), a markedly increased susceptibility was observed in NQO1−/− bladder cells to B[α]P-induced generation of reactive oxygen species (ROS), relative to wild-type bladder cells.

Bladder cells from 24 week-old female wild-type and NQO1−/− mice were incubated with 100 μM benzo(α)pyrene (B[α]P) in full media for 18 h followed by 1 h DCF (10 μM) and 1 h media recovery, and then visualized under coverslips via microscopy, where ROS-positive cells fluoresce green. FIG. 3C is a bar graph 320 that illustrates example stress signaling dependence on genotype and stress type. The horizontal axis 322 indicates stress type and the vertical axis 324 indicates the count of ROS-positive cells normalized to wild-type values and expressed as relative positive ROS reactive dye DCF positive cells to represent stress signaling. The open bars 326 indicate data for wild-type mice and the filled bars 328 indicate data for NQO1−/− mice. Error bars indicate the standard deviation. On the horizontal axis 322, Acetone serves as a control stress type and B[α]P represents high stress. Graph 320 shows that NQO1−/− bladder tissue exhibits a tendency towards significantly stronger stress than does wild-type. The increased stress signaling is over 95% likely to be non-random (P<0.05).

This increased stress signaling might explain the profound loss of adhesion of the luminal epithelial layer, exposed to the harsh environment of the bladder lumen, in morphological examinations. In addition, NQO1−/− bladder showed increase in PCNA, ICAM and NF-kB, as compared with wild-type bladder. These increases appear to be in response to epithelial layer damage as an attempt to restore normalcy.

Increased caspase expression and activity in bladders from NQO1−/− mice. The analysis also showed an increase in caspase 3 activity and tumor suppressor p53 in 24-weeks old NQO1−/− mice as compared to age matched wild-type mice. Bladders from 24 week old female wild-type and NQO1−/− mice were excised and lysed in either RIPA buffer (for Western blots) or Promega Passive Lysis buffer (caspase 3 activity assay) and analyzed. FIG. 3D shows four images of Western blot analysis of cleaved caspase 3 protein as marker of apoptosis. Higher cleaved caspase 3 in panel 330b indicates higher apoptosis in NQO1−/− mice, as compared to wild type mice in panel 332.

FIG. 3E is a bar graph 340 that illustrates example caspase 3 activity dependence on genotype. The horizontal axis 342 indicates a 24 week old mouse and the vertical axis 344 indicates the caspase 3 activity normalized to wild-type values. The open bars 346 indicate data for wild-type mice and the filled bars 348 indicate data for NQO1−/− mice. Graph 340 shows a significant increase in caspase 3 activity in 24-weeks old NQO1−/− mice as compared to age matched wild-type mice. The error bars indicate standard deviation among the samples averaged and the increased caspase 3 activity is over 95% likely to be non-random (P<0.05).

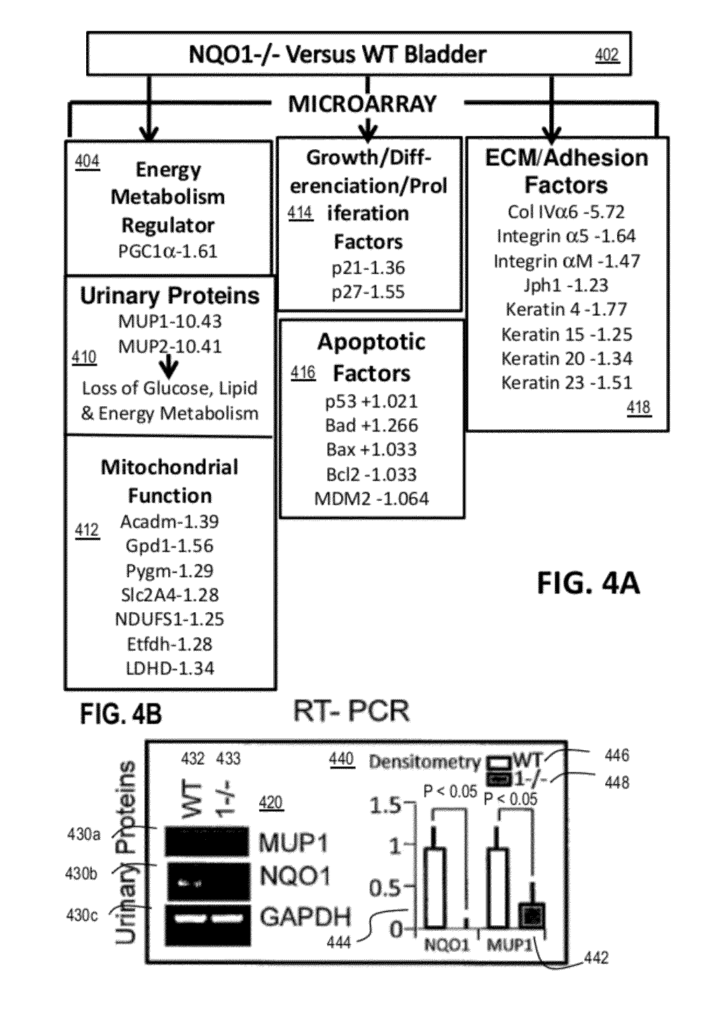

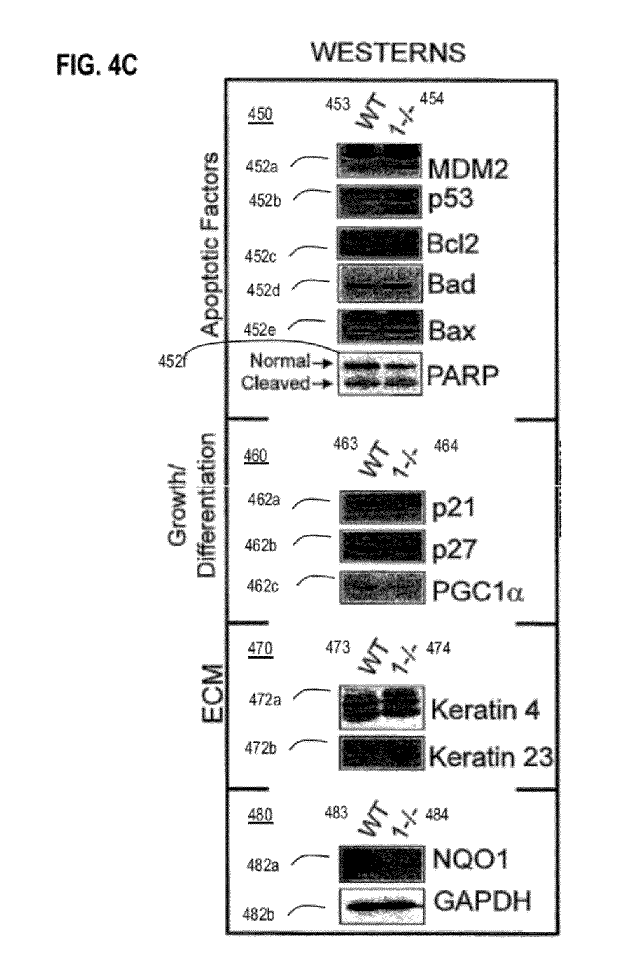

Disruption of several regulatory cascades in PBS-positive bladder tissue. FIG. 4A through FIG. 4C are graphs that illustrate example modulation of signaling cascades in a mouse model for PBS compared to wild-type mouse, according to an embodiment.

Microarray analysis was performed to discover possible molecular explanations for these observed morphological and apoptotic changes. Bladders from 24 week old female wild-type and NQO1−/− mice were excised and total RNA was purified using Qiagen RNEasy kit with DNAse treatment. Aliquots were analyzed via gene microarray and results were analyzed with statistical and pathways analysis software. FIG. 4A is a diagram that illustrates example microarray changes observed between bladder tissue of wild type and NQO1−/− genotypes, as indicated in panel 402. The microarray components are divided into separate panels representing different pathways. Panel 404 encloses the microarray component PGC1α protein associated with energy metabolism, which decreases in the NQO1−/− genotype. Panel 410 encloses the microarray components MUP1 and MUP2 associated with urinary proteins, both of which decrease in the NQO1−/− genotype. Panel 412 encloses the microarray components associated with mitochondrial function, all of which decrease in the NQO1−/− genotype. Panel 414 encloses the microarray components associated with growth/proliferation/differentiation factors, both of which decrease in the NQO1−/− genotype. Panel 418 encloses the microarray components associated with extracellular matrix/adhesion factors, all of which decrease in the NQO1−/− genotype.

In contrast to the other panels, panel 416 encloses the microarray components associated with apoptotic factors, most of which increase in the NQO1−/− genotype.

Thus, down-regulation of several cellular signaling pathways, including mitochondrial function, cellular growth/proliferation/differentiation, anti-apoptosis, and extracellular matrix/adhesion was observed. Interestingly, NQO1−/− mice bladder showed a significant down regulation of PGC1α a well-characterized strong regulator of energy metabolism and a significant down regulation of major urinary proteins (MUP1 and MUP2), a class of proteins which have as yet been poorly characterized, but have been shown to regulate energy metabolism in cells. Microarray data also showed down regulation of anti-apoptotic and up regulation of apoptotic factors in NQO1−/− mice, as compared to wild type mice bladder tissues.

RT PCR and/or Western blotting was performed to verify changes in most relevant genes and determined that there was, in fact, a significant down regulation of MUPs, PGC1α, and anti-apoptotic/growth and differentiation factors such as Bcl-2, p21, and p27. FIG. 4B shows RT PCR analysis that illustrates example decrease in major urinary protein expression in NQO1−/− bladder tissue 433 compared to wild type 432. Total RNA from 24 week old bladders was analyzed via RT-PCR using primers against MUP1 in image 430a, NQO1 in image 430b, and GAPDH control in image 430c. FIG. 4B includes bar graph 440, for which the horizontal axis 442 indicates the primer target and the vertical axis indicates the amount of RNA as indicated by the density of the image normalized to the wild type. The open bars 446 indicate data for wild-type mice and the filled bars 448 indicate data for NQO1−/− mice. The error bars indicate the standard deviation among the samples averaged and the decreased occurrence of the RNA for the urinary proteins is over 95% likely to be non-random (P<0.05).

FIG. 4C shows Western blotting analysis that illustrates example decrease in protein expression of key regulating factors in NQO1−/− bladder. Bladders from 24 week old mice were excised and homogenized in RIPA buffer, and aliquots were separated via SDS-PAGE gel and probed as shown in FIG. 4C with antibodies against MDM2 in image 452a, p53 in image 452b, Bcl-2 in image 452c, Bad in image 452d, Bax in image 452e, PARP in image 452f, p21 in image 462a, p27 in image 462b, PGC1α in image 462c, Keratin 4 in image 472a and Keratin 23 in image 472b, NQO1 in image 482 a, and GAPDH control in image 482b. FIG. 4C shows large up-regulation in pro-apoptotic factors in panel 450 such as p53, Bad, and Bax. Additionally, there was a strong down-regulation in extracellular matrix/adhesion-related factors in panel 470 such as Keratins 4 and 23. These results suggest a set of molecular signaling cascades, including energy metabolism and mitochondrial function, which may lead directly to apoptosis and ECM disruption, thus explaining the observed morphological aberrations in PBS-positive NQO1−/− mouse bladder tissues.

Ultrasctructural changes in NQO1-null bladder mitochondria. After determining that the loss of NQO1 in the bladder leads to problems in energy metabolism signaling, possible changes in mitochondria, which may explain a decrease in mitochondrial function, were investigated. Tunneling electron microscopy (TEM) techniques were used to photograph the full thickness of bladders from 24 week old wild-type and NQO1−/− female mice and the field of view environment was the luminal epithelium where the previously-observed lesions were present. In the luminal epithelium the mitochondria were measured and differences between wild-type and NQO1−/− tissues were recorded. There were striking ultrastructural differences in the mitochondria of NQO1−/− bladders.

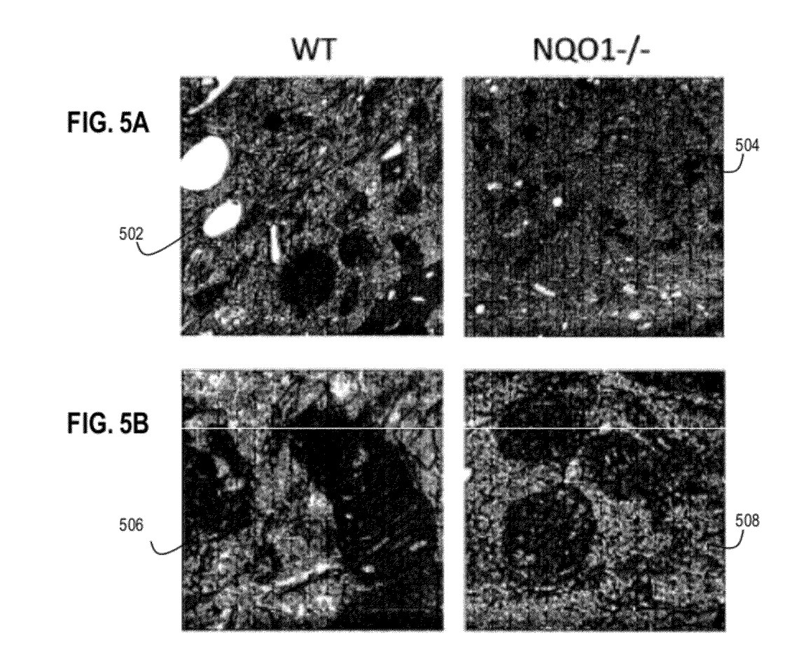

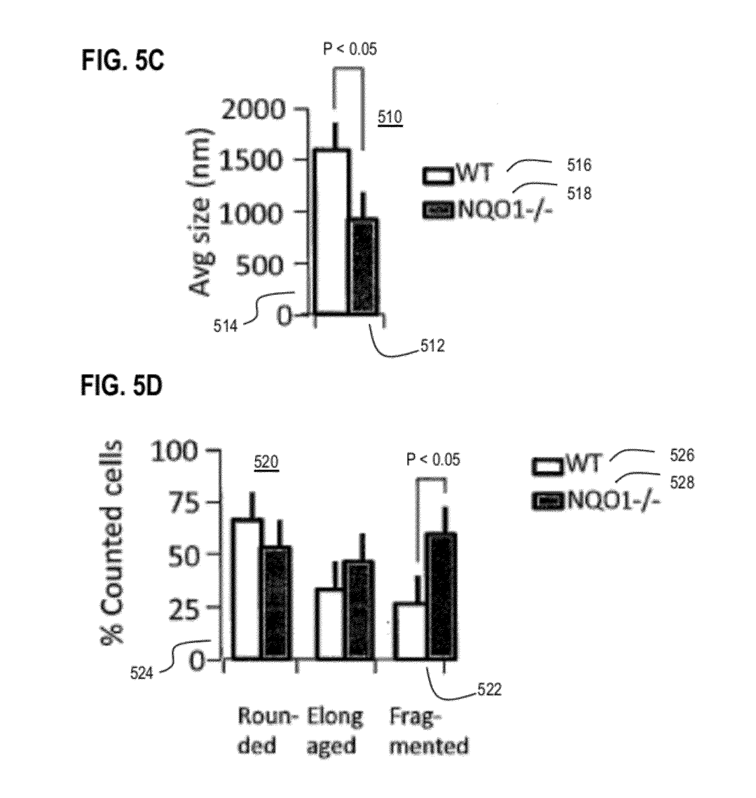

FIG. 5A through FIG. 5D are micrographs or graphs that illustrate example mitochondrial aberrations in a mouse model for PBS compared to wild-type mouse, according to an embodiment. Bladders from 24 week old wild-type and NQO1−/− mice were fixed in glutaraldehyde/formaldehyde, embedded in epoxy resin, and sections containing luminal epithelium were photographed via tunneling electron microscopy (TEM) at 4400× magnification. FIG. 5A illustrates example tunneling electron microscopy micrograph 502 of a wild type mouse and example tunneling electron microscopy micrograph 504 of a NQO1−/− mouse, showing a significant decrease in average size, as compared to wild-type.

FIG. 5C is a bar graph 510 that illustrates example mitochondrial size dependence on genotype. The horizontal axis 512 indicates a 24 week old mouse and the vertical axis 514 indicates the average mitochondrial size in nanometers (m, 1 nm=10⁻⁹ meters). The open bars 516 indicate data for wild-type mice and the filled bars 518 indicate data for NQO1−/− mice. Graph 510 shows a significant decrease of mitochondrial size in 24-weeks old NQO1−/− mice as compared to age matched wild-type mice. The error bars indicate standard deviation among the samples averaged and the decreased size is over 95% likely to be non-random (P<0.05)

Resin-embedded bladder sections were photographed using tunneling electron microscopy at 30000× magnification to produce FIG. 5B. FIG. 5B illustrates example tunneling electron microscopy micrograph 506 of a wild type mouse and example tunneling electron microscopy micrograph 508 of a NQO1−/− mouse, showing an increased roundedness of mitochondria in NQO1−/− bladder epithelium, as compared to wild-type. The mitochondria of bladders lacking NQO1 expression tended to be rounded, as opposed to the typical elliptical/bacillus shape found in most tissues and observed in wild-type bladder mitochondria. The most striking observation, however, was that in many NQO1−/− mitochondria, both an increase in fragmented outer mitochondrial membranes and a noticeable decrease or lack of discernable cristae structure within the mitochondrions were observed.

FIG. 5D is a bar graph 520 that illustrates example mitochondrial shape dependence on genotype. The horizontal axis 522 indicates shape observed in a 24 week old mouse and the vertical axis 524 indicates the number of cells counted with the specified shape in each field of view. The open bars 526 indicate data for wild-type mice and the filled bars 528 indicate data for NQO1−/− mice. Tunneling electron microscopy micrographs of resin-embedded bladder sections were measured for shape (rounded vs elongated) and fragmentation of inner cristae. Measurements are averages for mitochondria found in at least 10 visual fields. Graph 520 shows a significant increase of fragmentation inside mitochondria in 24-weeks old NQO1−/− mice as compared to age matched wild-type mice. The error bars indicate standard deviation among the samples averaged and the increased fragmentation is over 95% likely to be non-random (P<0.05).

These structural aberration in mitochondria may explain observed changes in energy metabolism and may be explained by upstream loss of gene expression.

NQO1 regulation of PGC1α via direct interaction. Earlier, it was found that NQO1 directly interacts with p53 and p63 and protect these factors against 20S proteasomal degradation (e.g., see Gong X., L. Kole, K. Iskander, et al., “NRH:quinone oxidoreductase 2 and NAD(P)H:quinone oxidoreductase 1 protect tumor suppressor p53 against 20S proteasomal degradation leading to stabilization and activation of p53,” Cancer Res v67, pp 5380-5388, 2007; and Patrick B. A., X. Gong, A. K. Jaiswal, “Disruption of NAD(P)H:quinone oxidoreductase 1 gene in mice leads to 20S proteasomal degradation of p63 resulting in thinning of epithelium and chemical-induced skin cancer,” Oncogene; v30, pp 1098-1107, 2011). Therefore, we investigated whether signs of mitochondrial energy metabolism which were previously observed could be due to a lack of protection of metabolism-signaling factors by NQO1. We selected PGC1α for further studies on mechanism of NQO1 control of factors because of its established role in control of energy metabolism. We also included two established regulators of PGC1α expression, cAMP response element-binding (CREB) and AMP-activated protein kinase (AMPK) (e.g., see Fernandez-Marcos P. J., J. Auwerx, “Regulation of PGC-1a, a nodal regulator of mitochondrial biogenesis,” Am J Clin Nutr v93, pp 8845-8890, 2011) in our studies since PGC1α RNA was down regulated in bladders from NQO1−/− mice in microarray analysis (see FIG. 4A). Western analysis demonstrated significant decrease not only in PGC1α but also its regulators CREB and AMPK in bladders from NQO1−/− mice, as compared to wild type mice (FIG. 4C). We also used human bladder carcinoma cell line T24 for NQO1 control of PGC1α studies since it expresses detectable amounts of NQO1, PGC1α, CREB and AMPK.

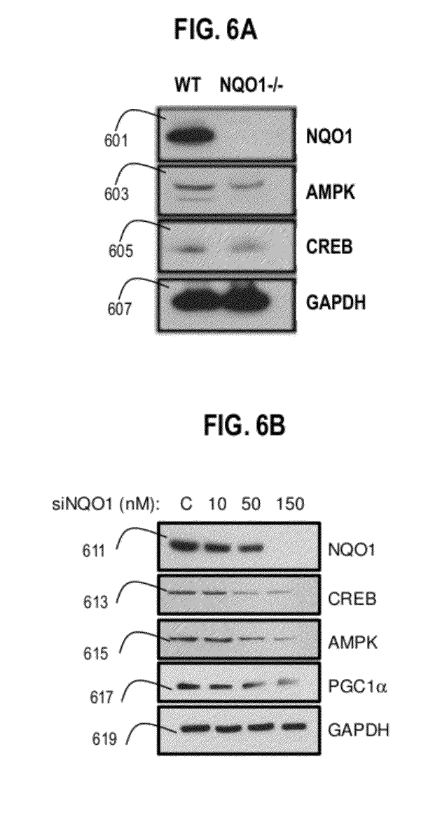

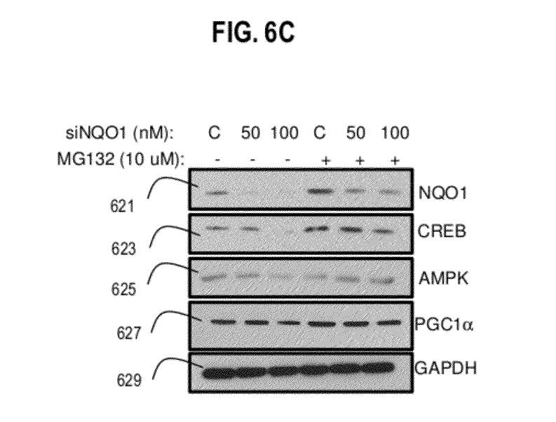

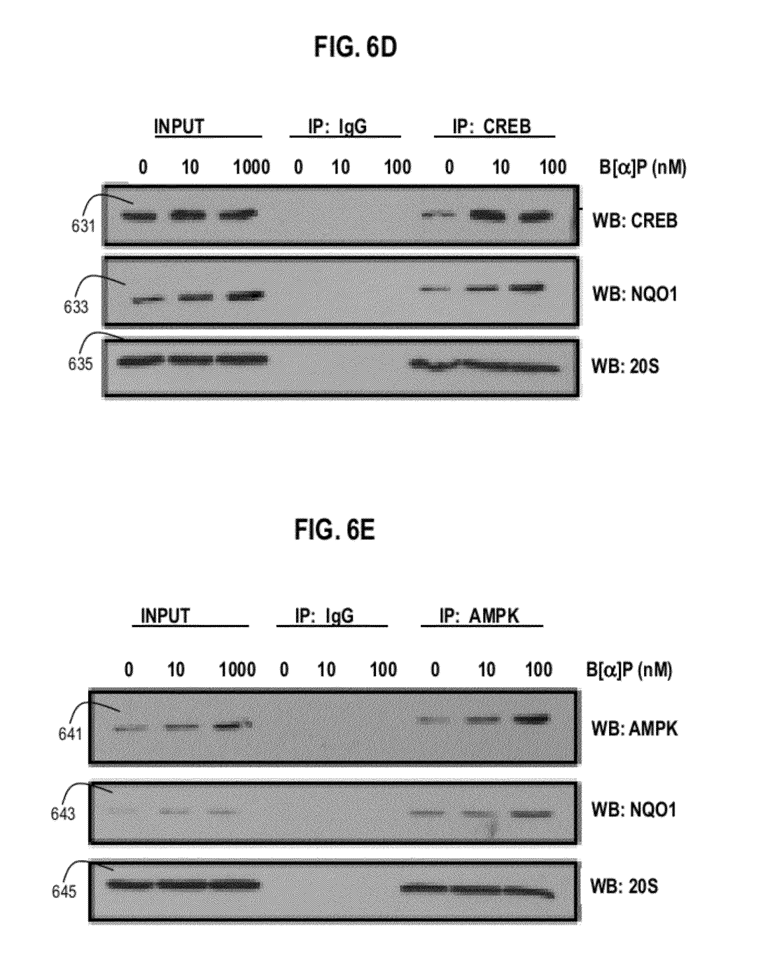

FIG. 6A through FIG. 6E are Western blot images that illustrate example NQO1 regulation of energy metabolic regulator factor PGC1α in a bladder of a mouse model for PBS compared to wild-type mouse, according to an embodiment. FIG. 6A is a set of Western blot images that illustrate example relative occurrence of proteins in bladders from wild type mice (left) and NQO1−/− mice (right). Bladders from wild type and NQO1−/− mice were immunoblotted and probed with antibodies as indicated: for NQO1 in blot image 601; for AMPK in blot image 603; for CREB in blot image 605; and, for control GAPDH in blot image 607. FIG. 6B is a set of Western blot images that illustrate example relative occurrence of proteins in T24 human bladder carcinoma cells transfected with control (column C, 100 nM control siRNA) or NQO1-specific siRNA (columns 10, 50 and 150 nM NQO1 siRNA) for 24 h and then immunoblotted with indicated antibodies: for NQO1 in blot image 611; for CREB in blot image 613; for AMPK in blot image 615; for PGC1α in blot image 617; and, for control GAPDH in blot image 619. FIG. 6C is a set of Western blot images that illustrate example relative occurrence of proteins in T24 human bladder carcinoma cells transfected with control (column C, 100 nM control siRNA) or NQO1-specific siRNA (columns 50 and 100 nM NQO1 siRNA) for 24 h and then incubated for 6 h with vehicle (signified by -) or proteasome inhibitor MG-132 (signified by +) before lysis and separation via SDS-PAGE. The samples were probed with the antibodies indicated: for NQO1 in blot image 621; for CREB in blot image 623; for AMPK in blot image 625; for PGC1α in blot image 627; and, for control GAPDH in blot image 629. FIG. 6D and FIG. 6E are sets of Western blot images that illustrate example relative occurrence of proteins in T24 cells that were incubated for 16 h with B[α]P at concentrations of 0, 10 and 100 nanoMoles (nM, 1 nM=10⁻⁹ Moles) and then immunoprecipitated with antibodies against CREB (in FIG. 6D) and AMPK (in FIG. 6E) overnight. IgG was used as immunoprecipitation control. Samples were separated via SDS-PAGE and probed with antibodies listed: for CREB in blot image 631 and 641; for NQO1 in blot image 633 and 643 and for 20S proteasome in blot image 635 and 645.

siRNA targeted NQO1 knockdown studies were used to determine that CREB, AMPK and PGC1α are all significantly regulated by NQO1 (FIG. 6B). siRNA mediated inhibition of NQO1 led to significant decreases in CREB, AMPK and PGC1α (FIG. 6B). Furthermore it was shown that CREB and AMPK but not PGC1α proteins were rescued from down regulation when proteasome activity was inhibited by MG-132 (FIG. 6C), suggesting that proteasomal degradation is a significant route of down regulation of CREB and AMPK proteins and that NQO1 expression is necessary to prevent that degradation.

Immunoprecipitation studies were performed to determine whether NQO1 can directly interact with CREB and AMPK, or PGC1α and whether that interaction is affected by known NQO1 inducer and stress agent benzopyrene (B[α]P). While no interaction was found with PGC1α directly, it was found that NQO1 is capable of binding to both CREB and AMPK in T24 and that such interaction appears to be increased during stress conditions leading to stabilization of those factors (FIGS. 6D and 6E). Immunoprecipitation studies also showed 20S proteasome interaction with CREB and AMPK that more or less did not change with benzo(a)pyrene stress (FIGS. 6D and 6E).

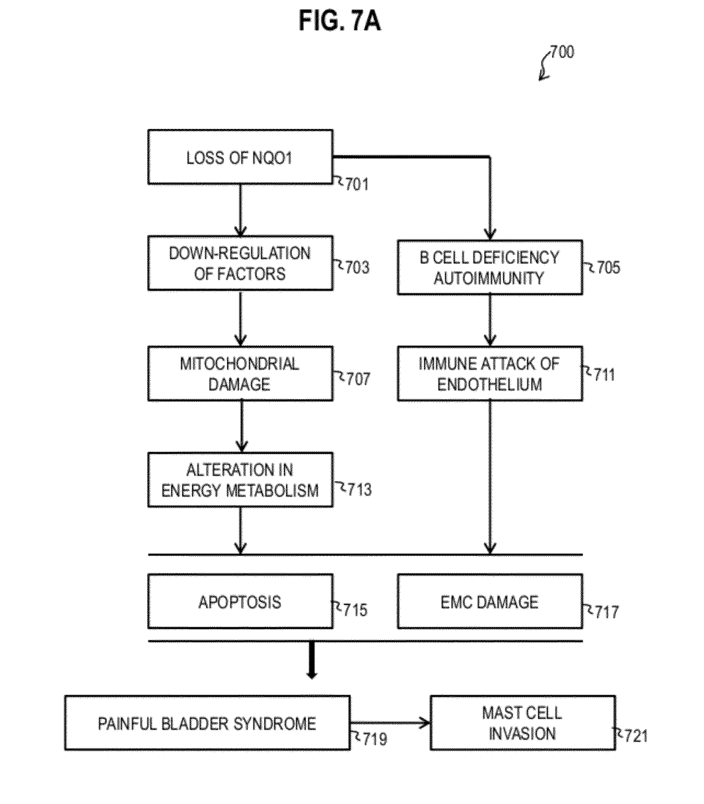

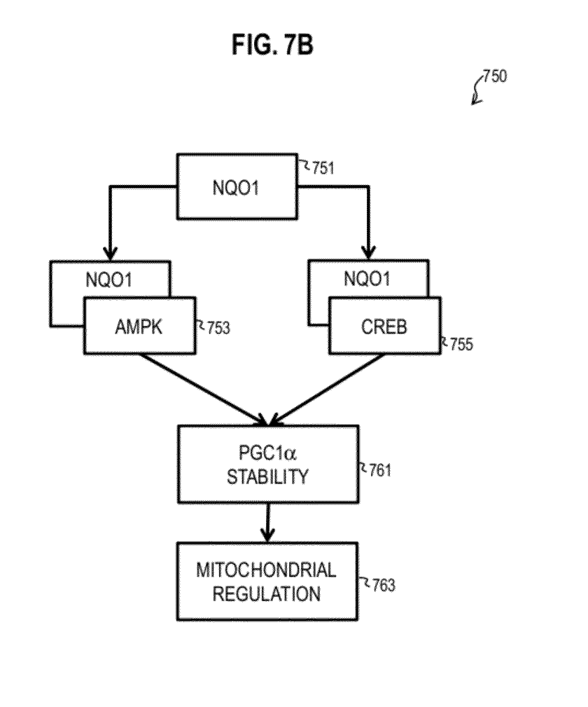

FIG. 7A and FIG. 7B are flow charts that illustrate model pathways of PBS pathology in a mouse model for PBS, according to an embodiment. Based on the above results we constructed a model for NQO1 protection against PBS (FIG. 7A) and NQO1 control of energy metabolism regulation of PGC1α via protection of AMPK/CREB from proteasomal degradation (FIG. 7B).

As shown in FIG. 7A, in step 701, loss of NQO1, e.g., by the SNP in the NQO1 gene, leads to down-regulation factors (identified above) in step 703; and, leads to B cell deficiency autoimmunity in step 705 (e.g., see Iskander K et al., J. Biol. Chem. v281, pp 30917-30924, 2006). The down-regulation of step 705 leads to mitochondrial damage in step 707 and alteration in energy metabolism in step 713. The B cell deficiency autoimmunity in step 705 leads to immune system attack of the endothelium in step 711. Steps 705 and 711 together cause apoptosis in step 715 and EMC damage in step 717. These steps result in PBS in step 719 and mast cell invasion in step 721.

As shown in FIG. 7B, in step 751, presence of NQO1, e.g., from the reference NQO1 gene, leads to protection of AMPK in step 753 and protection of CREB in step 755, Both these factors lead to increased PGC1α stability in step 761 and mitochondrial regulation in step 763.

The pathways shown in FIG. 7A and FIG. 7B, suggest points at which the genetic risk of PBS can be mitigated. For example, by administering activators to up-regulate the factors down-regulated in step 703, by activating energy metabolism regulators to mitigate step 713, by reducing B cell deficiency to avoid the autoimmunity of step 705, or by up-regulating AMPK or CREB, directly or indirectly.

### 2.2 Human Embodiment

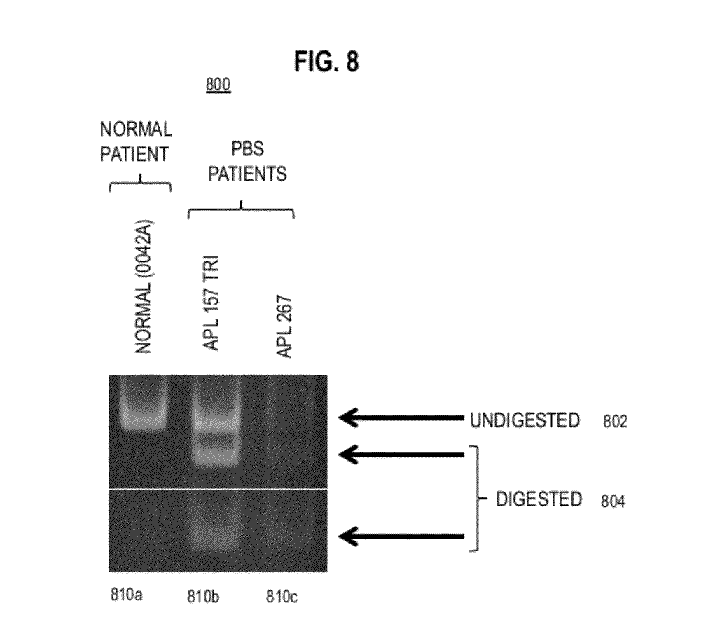

FIG. 8 is a photograph 800 that illustrates example genotyping of normal human and human PBS patients; according to an embodiment. DNA was PCR amplified, digested with Hinf I and separated on agarose gel. The target NQO1 gene is known to be in region 22 of chromosome 16 (16q22) of the human.

Normal DNA is not digested by Hinf I. Mutation of the NQO1 gene creates an Hinf I site and such mutated DNA is digested. After exposure to Hinf 1, normal NQO1 gene propagates in column 810a, a target NQO1 gene from a first human PBS patient propagates in column 810b, a target NQO1 gene from a second human PBS patient propagates in column 810c. Both PBS patients have one mutated NQO1 allele and DNA from each PBS patient was digested with Hinf I, as indicted by the smaller size and faster propagation to the region of the gel labeled digested 804. The normal patient had no mutated NQO1 allele and was not digested, as indicted by the larger size and slower propagation to the region of the gel labeled undigested 802.

This demonstrates that the genetic marker for PBS in mice is also the genetic marker for PBS in humans. The presence of a homozygous C-T mutation at position 609 of region 22 of human chromosome 16 is known to result in a loss of NQO1 protein and activity. The homozygous mutation represents a polymorphism in NQO1 which may be of significance since its prevalence in various populations has been reported to be between 6% and 17%. Recent data suggest that the human homozygous gentotype for this SNP occurs in about 3% of the population, and that the human heterozygous gentotype occurs in about 25% of the population, e.g., at about ten times the rate of the homozygous genotype. Since both genotypes are at above average risk, a significant portion of the population is affected. While NQO1 inducers offer potential treatment for the heterozygous gentotype that produces at least some NQO1, different treatments are indicated for the homozygous gentotype.

### 3. Experimental Methods

Methods of isolating nucleic acid molecules from a biological sample are routine, for example using PCR to amplify the molecules from the sample, or by using a commercially available kit to isolate DNA. Nucleic acid molecules isolated from PBMCs or any other biological sample can be amplified using routine methods to form nucleic acid amplification products. Preparation of Nucleic Acids for Analysis: Nucleic acid molecules can be prepared for analysis using any technique known to those skilled in the art. Generally, such techniques result in the production of a nucleic acid molecule sufficiently pure to determine the presence or absence of one or more variations at one or more locations in the nucleic acid molecule. Such techniques are described for example, in Sambrook, et al., Molecular Cloning: A Laboratory Manual (Cold Spring Harbor Laboratory, New York) (1989), and Ausubel, et al., Current Protocols in Molecular Biology (John Wiley and Sons, New York) (1997), incorporated herein by reference.

Amplification of nucleic acid molecules: Optionally, the nucleic acid samples obtained from the subject are amplified prior to detection. Target nucleic acids are amplified to obtain amplification products, including sequences from a tag SNP, can be amplified from the sample prior to detection. Typically, DNA sequences are amplified, although in some instances RNA sequences can be amplified or converted into cDNA, such as by using RT PCR.

Methods for labeling nucleic acid molecules so they can be detected are well known. Examples of such labels include non-radiolabels and radiolabels. Non-radiolabels include, but are not limited to an enzyme, chemiluminescent compound, fluorescent compound (such as FITC, Cy3, and Cy5), metal complex, hapten, enzyme, colorimetric agent, a dye, or combinations thereof. Radiolabels include, but are not limited to, 125I, 32P and 35S. For example, radioactive and fluorescent labeling methods, as well as other methods known in the art, are suitable for use with the present disclosure. In one example, primers used to amplify the subject's nucleic acids are labeled (such as with biotin, a radiolabel, or a fluorophore). In another example, amplified target nucleic acid samples are end-labeled to form labeled amplified material. For example, amplified nucleic acid molecules can be labeled by including labeled nucleotides in the amplification reactions.

Nucleic acid molecules corresponding to one or more tag SNPs or haplotype blocks including the tag SNP can also be detected by hybridization procedures using a labeled nucleic acid probe, such as a probe that detects only one alternative allele at a marker locus. Most commonly, the target nucleic acid (or amplified target nucleic acid) is separated based on size or charge and transferred to a solid support. The solid support (such as membrane made of nylon or nitrocellulose) is contacted with a labeled nucleic acid probe, which hybridizes to it complementary target under suitable hybridization conditions to form a hybridization complex.

Hybridization conditions for a given combination of array and target material can be optimized using methods known to one of skill in the art (see U.S. Pat. No. 5,981,185). Once the target nucleic acid molecules have been hybridized with the labeled probes, the presence of the hybridization complex can be analyzed, for example by detecting the complexes.

Methods for detecting hybridized nucleic acid complexes are well known in the art. In one example, detection includes detecting one or more labels present on the oligonucleotides, the target (e.g., amplified) sequences, or both. Detection can include treating the hybridized complex with a buffer and/or a conjugating solution to effect conjugation or coupling of the hybridized complex with the detection label, and treating the conjugated, hybridized complex with a detection reagent. In one example, the conjugating solution includes streptavidin alkaline phosphatase, avidin alkaline phosphatase, or horseradish peroxidase. Specific, non-limiting examples of conjugating solutions include streptavidin alkaline phosphatase, avidin alkaline phosphatase, or horseradish peroxidase. The conjugated, hybridized complex can be treated with a detection reagent. In one example, the detection reagent includes enzyme-labeled fluorescence reagents or calorimetric reagents. In one specific non-limiting example, the detection reagent is enzyme-labeled fluorescence reagent (ELF) from Molecular Probes, Inc. (Eugene, Oreg.). The hybridized complex can then be placed on a detection device, such as an ultraviolet (UV) transilluminator (manufactured by UVP, Inc. of Upland, Calif.). The signal is developed and the increased signal intensity can be recorded with a recording device, such as a charge coupled device (CCD) camera (manufactured by Photometrics, Inc. of Tucson, Ariz.). In particular examples, these steps are not performed when radiolabels are used. In particular examples, the method further includes quantification, for instance by determining the amount of hybridization.

Allele Specific PCR: Allele-specific PCR differentiates between target regions differing in the presence of absence of a variation or polymorphism. PCR amplification primers are chosen based upon their complementarity to the target sequence, such as nucleic acid sequence in a haplotype block including a tag SNP, a specified region of an allele including a tag SNP, or to the tag SNP itself. The primers bind only to certain alleles of the target sequence. This method is described by Gibbs, Nucleic Acid Res. 17:12427 2448, 1989, herein incorporated by reference.

Allele Specific Oligonucleotide Screening Methods: Further screening methods employ the allele-specific oligonucleotide (ASO) screening methods (e.g. see Saiki et al., Nature 324:163-166, 1986). Oligonucleotides with one or more base pair mismatches are generated for any particular allele or haplotype block. ASO screening methods detect mismatches between one allele (or haplotype block) in the target genomic or PCR amplified DNA and the other allele (or haplotype block), showing decreased binding of the oligonucleotide relative to the second allele (i.e. the other allele) oligonucleotide. Oligonucleotide probes can be designed that under low stringency will bind to both polymorphic forms of the allele, but which at high stringency, only bind to the allele to which they correspond. Alternatively, stringency conditions can be devised in which an essentially binary response is obtained, i.e., an ASO corresponding to a variant form of the target gene will hybridize to that allele (haplotype block), and not to the reference allele (haplotype block).

Ligase Mediated Allele Detection Method: Ligase can also be used to detect point mutations, such as the tag SNPs disclosed herein, in a ligation amplification reaction (e.g. as described in Wu et al., Genomics 4:560-569, 1989). The ligation amplification reaction (LAR) utilizes amplification of specific DNA sequence using sequential rounds of template dependent ligation (e.g. as described in Wu, supra, and Barany, Proc. Nat. Acad. Sci. 88:189-193, 1990).

Denaturing Gradient Gel Electrophoresis: Amplification products generated using the polymerase chain reaction can be analyzed by the use of denaturing gradient gel electrophoresis. Different alleles (haplotype blocks) can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (T.sub.M). Melting domains are at least 20 base pairs in length, and can be up to several hundred base pairs in length.

Non-gel Systems: Other possible techniques include non-gel systems such as TaqMan™ (Perkin Elmer). In this system oligonucleotide PCR primers are designed that flank the mutation in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete (there is a mismatch of some form) the cleavage of the dye does not take place. Thus only if the nucleotide sequence of the oligonucleotide probe is completely complimentary to the template molecule to which it is bound will quenching be removed. A reaction mix can contain two different probe sequences each designed against different alleles that might be present thus allowing the detection of both alleles in one reaction.

Non-PCR Based Allele detection: The identification of a DNA sequence can be made without an amplification step, based on polymorphisms including restriction fragment length polymorphisms in a subject and a control, such as a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes can be labeled directly or indirectly, such that by assaying for the presence or absence of the probe, one can detect the presence or absence of the target sequence. Direct labeling methods include radioisotope labeling, such as with .sup.32P or .sup.35S. Indirect labeling methods include fluorescent tags, biotin complexes which can be bound to avidin or streptavidin, or peptide or protein tags. Visual detection methods include photoluminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

### 4. Arrays

Certain embodiments are directed to an array for detecting a genetic predisposition to PBS in a human subject. The array contains probes complementary to at least one tag single nucleotide polymorphism. Each of the tag single nucleotide polymorphisms is associated with a risk allele for PBS and the probes complementary to the tag single nucleotide polymorphism hybridize specifically to the nucleotide.

It will be readily apparent to one skilled in the art that the exact formulation of probes on an array is not critical as long as the user is able to select probes for inclusion on the array that fulfill the function of hybridizing to a protective allele or probes that are complementary to a tag SNP, wherein the tag SNPs is associated with a risk allele as defined herein. The array can be modified to suit the needs of the user. Thus, analysis of the array can provide the user with information regarding the number and/or presence of risk alleles and/or protective alleles in a given sample. The hybridization of a probe complementary to a risk allele in an array can indicate that the subject from whom the sample was derived is at an elevated risk for developing a disease such as PBS; or alternatively if it hybridizes to a protective allele the subject has a reduced risk.

A wide variety of array formats can be employed in accordance with the present disclosure. One example includes a linear array of oligonucleotide bands, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use (see U.S. Pat. No. 5,981,185). In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. Biaxially oriented polypropylene (BOPP) films are also suitable in this regard; in addition to their durability, BOPP films exhibit a low background fluorescence. In a particular example, the array is a solid phase, Allele-Specific Oligonucleotides (ASO) based nucleic acid array.

The array formats of the present disclosure can be included in a variety of different types of formats. A “format” includes any format to which the solid support can be affixed, such as microtiter plates, test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are known to those working in the field; a summary of suitable methods can be found in Matson et al., Anal. Biochem. 217:306-10, 1994. In one example, the oligonucleotides are synthesized onto the support using conventional chemical techniques for preparing oligonucleotides on solid supports (such as see PCT Publication No. WO 85/01051 and PCT Publication No. WO 89/10977, or U.S. Pat. No. 5,554,501).

A suitable array can be produced using automated means to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90.degree. to permit synthesis to proceed within a second (2.degree.) set of rows that are now perpendicular to the first set. This process creates a multiple-channel array whose intersection generates a plurality of discrete cells.

In particular examples, the oligonucleotide probes on the array include one or more labels, which permit detection of oligonucleotide probe:target sequence hybridization complexes.

### 5. Kits

Certain embodiments are directed to kits that can be used to detect a genetic predisposition to PBS or hypertensive end stage kidney disease. The disclosed kits can include a binding molecule, such as an oligonucleotide probe that selectively hybridizes to an allele of a haplotype block including a tag SNP. Alternatively or additionally, the kits can include one or more isolated primers or primer pairs for amplifying a target nucleic acid, such as a haplotype including a tag SNP. For example, the kit can include primers for amplifying a haplotype including tag SNPs, wherein the sequence includes the tag SNP associated with PBS.

The kit can further include one or more of a buffer solution, a conjugating solution for developing the signal of interest, or a detection reagent for detecting the signal of interest, each in separate packaging, such as a container. In another example, the kit includes a plurality of size-associated marker target nucleic acid sequences for hybridization with a detection array. The target nucleic acid sequences can include oligonucleotides such as DNA, RNA, and peptide-nucleic acid, or can include PCR fragments. The kit can also include instructions in a tangible form, such as written instructions or in a computer-readable format.

### 6. Pharmaceutical Formulations

The therapeutic agents may be present in the pharmaceutical compositions in the form of salts of pharmaceutically acceptable acids or in the form of bases. The therapeutic agents may be present in amorphous form or in crystalline forms, including hydrates and solvates. Preferably, the pharmaceutical compositions comprise a therapeutically effective amount.

Pharmaceutically acceptable salts of the therapeutic agents described herein include those salts derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate salts. Other acids, such as oxalic, while not in themselves pharmaceutically acceptable, may be employed in the preparation of salts useful as intermediates in obtaining pharmaceutically acceptable acid addition salts.

Salts derived from appropriate bases include alkali metal (e.g., sodium and potassium), alkaline earth metal (e.g., magnesium), ammonium and N⁺(C₁₋₄ alkyl)₄ salts. It is anticipated that some embodiment include the quaternization of any basic nitrogen-containing groups of the therapeutic agents disclosed herein. Water or oil-soluble or dispersible products may be obtained by such quaternization.

The therapeutic agents of some embodiments are also meant to include all stereochemical forms of the therapeutic agents (i.e., the R and S configurations for each asymmetric center). Therefore, single enantiomers, racemic mixtures, and diastereomers of the therapeutic agents are within the scope of the invention. Also within the scope of the invention are steric isomers and positional isomers of the therapeutic agents. The therapeutic agents of some embodiments are also meant to include compounds which differ only in the presence of one or more isotopically enriched atoms. For example, therapeutic agents in which one or more hydrogens are replaced by deuterium or tritium, or the replacement of one or more carbons by ¹³C- or ¹⁴C-enriched carbon are within the scope of this invention.

In a preferred embodiment, the therapeutic agents of some embodiments are administered in a pharmaceutical composition that includes a pharmaceutically acceptable carrier, adjuvant, or vehicle. The term “pharmaceutically acceptable carrier, adjuvant, or vehicle” refers to a non-toxic carrier, adjuvant, or vehicle that does not destroy or significantly diminish the pharmacological activity of the therapeutic agent with which it is formulated. Pharmaceutically acceptable carriers, adjuvants or vehicles that may be used in the compositions of some embodiments encompass any of the standard pharmaceutically accepted liquid carriers, such as a phosphate-buffered saline solution, water, as well as emulsions such as an oil/water emulsion or a triglyceride emulsion. Solid carriers may include excipients such as starch, milk, sugar, certain types of clay, stearic acid, talc, gums, glycols, or other known excipients. Carriers may also include flavor and color additives or other ingredients. The formulations of the combination of some embodiments may be prepared by methods well-known in the pharmaceutical arts and described herein. Exemplary acceptable pharmaceutical carriers have been discussed above. An additional carrier, Cremophor™, may be useful, as it is a common vehicle for Taxol.

The pharmaceutical compositions of the some embodiments preferably administered orally, preferably as solid compositions. However, the pharmaceutical compositions may be administered parenterally, by inhalation spray, topically, rectally, nasally, buccally, vaginally or via an implanted reservoir. Sterile injectable forms of the pharmaceutical compositions may be aqueous or oleaginous suspensions. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

The pharmaceutical compositions employed in some embodiments may be orally administered in any orally acceptable dosage form, including, but not limited to, solid forms such as capsules and tablets. In the case of tablets for oral use, carriers commonly used include microcrystalline cellulose, lactose and corn starch. Lubricating agents, such as magnesium stearate, are also typically added. When aqueous suspensions are required for oral use, the active ingredient may be combined with emulsifying and suspending agents. If desired, certain sweetening, flavoring or coloring agents may also be added.

The pharmaceutical compositions employed in the some embodiments may also be administered by nasal aerosol or inhalation. Such pharmaceutical compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Should topical administration be desired, it can be accomplished using any method commonly known to those skilled in the art and includes but is not limited to incorporation of the pharmaceutical composition into creams, ointments, or transdermal patches.

The passage of agents through the blood-brain barrier to the brain can be enhanced by improving either the permeability of the agent itself or by altering the characteristics of the blood-brain barrier. Thus, the passage of the agent can be facilitated by increasing its lipid solubility through chemical modification, and/or by its coupling to a cationic carrier. The passage of the agent can also be facilitated by its covalent coupling to a peptide vector capable of transporting the agent through the blood-brain barrier. Peptide transport vectors known as blood-brain barrier permeabilizer compounds are disclosed in U.S. Pat. No. 5,268,164. Site specific macromolecules with lipophilic characteristics useful for delivery to the brain are disclosed in U.S. Pat. No. 6,005,004.

Examples of routes of administration comprise parenteral, e.g., intravenous, intradermal, subcutaneous, inhalation, transdermal (topical), transmucosal, and rectal administration; or oral. Solutions or suspensions used for parenteral, intradermal, or subcutaneous application can comprise the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. Pharmaceutical compositions suitable for injection comprise sterile aqueous solutions (where water soluble) or dispersions and sterile powders for the extemporaneous preparation of sterile injectable solutions or dispersions. For intravenous administration, suitable carriers comprise physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the selected particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some cases, isotonic agents are included in the composition, for example, sugars, polyalcohols such as manitol, sorbitol, or sodium chloride. Prolonged absorption of an injectable composition can be achieved by including in the composition an agent that delays absorption, for example, aluminum monostearate or gelatin.

Sterile injectable solutions can be prepared by incorporating the active compound in the specified amount in an appropriate solvent with one or a combination of ingredients enumerated above, as needed, followed by filtered sterilization. Generally, dispersions are prepared by incorporating the active compound into a sterile vehicle that contains a basic dispersion medium and other ingredients selected from those enumerated above or others known in the art. In the case of sterile powders for the preparation of sterile injectable solutions, the methods of preparation comprise vacuum drying and freeze-drying which yields a powder of the active ingredient plus any additional desired ingredient from a previously sterile-filtered solution thereof.

Oral compositions generally comprise an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be comprised as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and comprise, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

### 7. Nucleotide Sequences

In alternative embodiments, one or more molecules include one or more of the nucleotide sequences described next.

Deoxyribonucleic acid (DNA) is a usually double-stranded long molecule that encodes other shorter molecules, such as proteins, used to build and control all living organisms. DNA is composed of repeating chemical units known as “nucleotides” or “bases.”

There are four bases: adenine, thymine, cytosine, and guanine, represented by the letters A, T, C and G, respectively. Adenine on one strand of DNA always binds to thymine on the other strand of DNA; and guanine on one strand always binds to cytosine on the other strand and such bonds are called base pairs. Any order of A, T, C and G is allowed on one strand, and that order determines the complementary order on the other strand. The actual order determines the function of that portion of the DNA molecule. Information on a portion of one strand of DNA can be captured by ribonucleic acid (RNA) that also comprises a chain of nucleotides in which uracil (U) replaces thymine (T). Determining the order, or sequence, of bases on one strand of DNA or RNA is called sequencing. A portion of length k bases of a strand is called a k-mer; and specific short k-mers are called oligonucleotides or oligomers or “oligos” for short.

It is known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” is known in the art to refer to the region between the translation initiation codon and the translation termination codon. It is also known in the art that variants can be produced through the use of alternative signals to start or stop transcription and that pre-mRNAs and mRNAs can possess more than one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites.

In the context of various embodiments, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of a nucleic acid is capable of hydrogen bonding with a nucleotide at the same position of a DNA or RNA molecule, then the nucleic acid and the DNA or RNA are considered to be complementary to each other at that position. The nucleic acid and the DNA or RNA are complementary to each other when a sufficient number of corresponding positions in each molecule are occupied by nucleotides that can hydrogen bond with each other. Thus, “specifically hybridizable” and “complementary” are terms that are used to indicate a sufficient degree of complementarity or precise pairing such that stable and specific binding occurs between the nucleic acid and the DNA or RNA target.

Various conditions of stringency can be used for hybridization as is described below. As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1 6.3.6, which is incorporated by reference. Aqueous and nonaqueous methods are described in that reference and either can be used. Specific hybridization conditions referred to herein are as follows: 1) low stringency hybridization conditions in 6.times.sodium chloride/sodium citrate (SSC) at about 45° C., followed by two washes in 0.2.times.SSC, 0.1% SDS at least at 50.degree C. (the temperature of the washes can be increased to 55° C. for low stringency conditions); 2) medium stringency hybridization conditions in 6.times.SSC at about 45° C., followed by one or more washes in 0.2.times.SSC, 0.1% SDS at 60° C.; 3) high stringency hybridization conditions in 6.times.SSC at about 45° C., followed by one or more washes in 0.2.times.SSC, 0.1% SDS at 65° C.; and preferably 4) very high stringency hybridization conditions are 0.5M sodium phosphate, 7% SDS at 65° C., followed by one or more washes at 0.2.times.SSC, 1% SDS at 65° C. Very high stringency conditions (4) are the preferred conditions and the ones that should be used unless otherwise specified.

Nucleic acids in the context of various embodiments include “oligonucleotides,” which refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. DNA/RNA chimeras are also included.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure; however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Oligonucleotides containing modified backbones or non-natural internucleoside linkages can be used. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides. Preferred modified oligonucleotide backbones include, for example, phosphorothioates, chiral phosphorothioates, phosphorodithioates, phosphotriesters, aminoalkyl-phosphotriesters, methyl and other alkyl phosphonates including 3-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference. Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

In some oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Some embodiments of some embodiments use oligonucleotides with phosphorothioate backbones and oligonucleosides with heteroatom backbones, and in particular —CH₂—NH—O—CH₂—, —CH₂—N(CH₃)—O—CH₂—[known as a methylene(methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—C H₂] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂).sub.nNH₂, O(CH₂)ₙCH₃, O(CH₂)ₙONH₂, and O(CH₂)ₙON[(CH₂).sub.nCH₃)]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy(2′-O—CH₂CH₂OCH₃, also known as 2′-O—(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylamino-ethoxyethoxy (also known in the art as 2′-O-dimethylamino-ethoxyethyl or 2′-DMAEOE), i.e., 2′-O—CH₂—O—CH₂—N(CH₂)₂, also described in examples hereinbelow.

A further modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2. LNAs and preparation thereof are described in WO 98/39352 and WO 99/14226.

Other modifications include 2′-methoxy(2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro(2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine. (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C.ident.C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of some embodiments. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

Another modification of the oligonucleotides for use in some embodiments involves chemically linking to the oligonucleotide one or more moieties or conjugates which enhance the activity, cellular distribution or cellular uptake of the oligonucleotide. The compounds of some embodiments can include conjugate groups covalently bound to functional groups such as primary or secondary hydroxyl groups. Conjugate groups of some embodiments include intercalators, reporter molecules, polyamines, polyamides, poly ethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of various embodiments, include groups that improve oligomer uptake, enhance oligomer resistance to degradation, and/or strengthen sequence-specific hybridization with RNA. Groups that enhance the pharmacokinetic properties, in the context of various embodiments, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et. al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid; e.g., di hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Oligonucleotides of some embodiments may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

It is not necessary for all positions in a given compound to be uniformly modified, and in fact more than one of the aforementioned modifications may be incorporated in a single compound or even at a single nucleoside within an oligonucleotide. “Chimeric” compounds or “chimeras,” in the context of various embodiments, are oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids.

The oligonucleotides used in accordance with various embodiments may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

### 8. Alternatives and Extensions

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items. elements or steps. Furthermore, the indefinite article “a” or “an” is meant to indicate one or more of the item, element or step modified by the article.

### 9. References

The entire contents of the following references are hereby incorporated by reference as if fully set forth herein, except for terminology that is inconsistent with the terminology used herein.
Fernandez-Marcos P. J., J. Auwerx, “Regulation of PGC-1a, a nodal regulator of mitochondrial biogenesis,” Am J Clin Nutr v93, pp 8845-8890, 2011.
Gong X., L. Kole, K. Iskander, et al., “NRH:quinone oxidoreductase 2 and NAD(P)H:quinone oxidoreductase 1 protect tumor suppressor p53 against 20S proteasomal degradation leading to stabilization and activation of p53,” Cancer Res v67, pp 5380-5388, 2007.
Hurst, R. E., R. M. Moldwin, S. G. Mulholland, “Bladder defense molecules, urothelial differentiation, urinary biomarkers, and interstitial cystitis,” Urology; v69, no. 4 Suppl., pp S17-S23, 2007.
Iskander K et al., J. Biol. Chem. v281, pp 30917-30924, 2006.
Kaspar J, S. K. Niture, A. K. Jaiswal. “Nrf2:INrf2 (Keap1) signaling in oxidative stress,” Free Rad Biol Med v47, pp 1304-1309, 2009.
Keay S., “Cell signaling in interstitial cystitis/painful bladder syndrome,” Cell Signal, v20, no. 12, pp 2174-2179, 2008.
Kilpatrick L. A., E. Ornitz, H. Ibrahimovic, et al., “Gating of sensory information differs in patients with interstitial cystitis/painful bladder syndrome,” J Urol, v184, no. 3, pp 958-963, 2010.
Park S, H. Zhao, M. R. Spitz, et al., “An association between NQO1 genetic polymorphism and risk of bladder cancer,” Mut Res v536, pp 131-137, 2003.
Patrick B. A., X. Gong, A. K. Jaiswal, “Disruption of NAD(P)H:quinone oxidoreductase 1 gene in mice leads to 20S proteasomal degradation of p63 resulting in thinning of epithelium and chemical-induced skin cancer,” Oncogene; v30, pp 1098-1107, 2011.
Radjendirane V, P. Joseph, Y. H. Lee, et al., “Disruption of the DT diaphorase (NQO1) gene in mice leads to increased menadione toxicity,” J Biol Chem v273, pp 7382-738, 1998.
Sant G. R, D. Kempuraj, J. E. Marchand, et al. “The mast cell in interstitial cystitis: role in pathophysiology and pathogenesis,” Urology; v69, no. 4 Suppl., pp 34-40, 2007.
Sanyal S, C. Ryk, P. J. De Verdier, et al., “Polymorphisms in NQO1 and the clinical course of urinary bladder neoplasms,” Scand J Urol Nephrol, v41, pp 182-190, 2007.
Ratliff T., C. G. Klutke, M. Hofineister, et al., “Role of the immune response in interstitial cystitis,” Clin Immunol Immunopathol, v74, pp 209-216, 1995.
Robbins M. T., J. DeBerry, T. J. Ness, “Chronic physiological stress enhances nociceptive processing in the urinary bladder in high-anxiety rats,” Physiol Behav, v91, pp 544-550, 2007.
Ross D, J. K. Kepa, S. L. Winski, et al., “NAD(P)H:quinone oxidoreductase 1 (NQO1): chemoprotection, bioactivation, gene regulation and genetic polymorphism,” Chem Biol Interact v129, pp 77-97, 2000.
Stone, A. R., K. B. Quattrocchi, C. H. Miller, et al., “Role of the immune system in interstitial cystitis,” Semin Urol, v9, pp 108-114, 1991.
Towbin H, T. Staehelin, J. Gordon, “Electrophoretic transfer of proteins from polyacrylamide gels to nitrocellulose sheets: procedure and some applications,” Proc Natl Acad Sci USA, v76, pp 4350-4354, 1979.

What is claimed is:

1. A method comprising:
obtaining a biological sample from a subject that does not display a symptom of Painful Bladder Syndrome (PBS);
analyzing the biological sample for a mutated form of NAD(P)H dehydrogenase [quinone], abbreviated NQO1, or a mutation of a NQO1 gene; and
determining a risk of PBS for the subject based on analyzing the biological sample.

2. A method as recited in claim 1, further comprising treating the subject with a therapeutically effective amount of an antioxidant if it is determined that the subject has an above normal risk of developing PBS.

3. A method as recited in claim 1, wherein:
obtaining the biological sample further comprises obtaining a deoxyribonucleic acid (DNA) sample from the subject;
analyzing the biological sample further comprises analyzing the DNA sample to detect the presence of a mutation in a NQO1 gene at a location where a reference NQO1 gene encodes for the enzyme NQO1; and
determining the risk of PBS further comprises determining that the subject has an above normal risk of developing PBS if the mutation in the NQO1 gene is detected in the DNA sample.

4. A method as recited in claim 3, wherein the mutation is in the NQO1 gene on human chromosome region 16q22.

5. A method as recited in claim 3, wherein the mutation is a single nucleotide polymorphism.

6. A method as recited in claim 5, wherein the single nucleotide polymorphism is a cytosine to thymine nucleotide substitution at position that leads to a proline to serine amino acid substitution at amino acid position 187 to produce a protein NQO1P187S.

7. A method as recited in claim 3, wherein determining that the subject has an above normal risk of developing Painful Bladder Syndrome further comprises:
determining that the subject is heterozygous in mutated NQO1 gene if the mutation in the NQO1 gene is detected in the DNA sample and the reference NQO1 gene is also detected in the DNA sample; and
determining that the subject is homozygous in the mutated NQO1 gene if the mutation in the NQO1 gene is detected in the DNA sample and if the reference NQO1 gene is not also detected in the DNA sample.

8. A method as recited in claim 1, wherein:
analyzing the biological sample further comprises analyzing the biological sample to detect the presence of a mutated form of NQO1; and
determining that the subject has an above normal risk of developing PBS if the mutated form of NQO1 is detected in the biological sample.

9. A method as recited in claim 8, wherein the mutated form of NQO1 is a proline to serine amino acid substitution at amino acid position 187 called protein NQO1P187S.

10. A method as recited in claim 8, wherein determining that the subject has an above normal risk of developing PBS further comprises:
determining that the subject is heterozygous in mutated NQO1 gene if the mutated form of NQO1 is detected in the biological sample and NQO1 is also detected in the biological sample; and
determining that the subject is homozygous in the mutated NQO1 gene if the mutated form of NQO1 is detected in the biological sample and if NQO1 is not also detected in the biological sample.

11. A method as recited in one of claims 7, further comprising treating the subject with a therapeutically effective amount of an inducer of NQO1 expression if it is determined that the subject is heterozygous in the mutated NQO1 gene.

12. A method as recited in claim 11, wherein the inducer of NQO1 expression is selected from a group comprising sulphoraphane, 4-(3)-tert-butyl-11-hydroxyanisole (BHA), tert-butylhydroquinone (t-BHQ), honokiol, and silibinin.

13. A method as recited in claim 7, further comprising treating the subject with a therapeutically effective amount of peroxisome proliferator-activated receptor gamma, coactivator 1 alpha, abbreviated PPARGC1A, if it is determined that the subject is homozygous in the mutated NQO1 gene.

14. A method as recited in claim 7, further comprising treating the subject with a therapeutically effective amount of an activator of an anti-apoptotic protein if it is determined that the subject is homozygous in the mutated NQO1 gene.

15. A method as recited in claim 14, wherein the activator of the anti-apoptotic protein is selected from a group comprising Bcl-xL and Bcl-2.

16. A method as recited in claim 7, further comprising treating the subject with a therapeutically effective amount of an inhibitor of an apoptotic proteins if it is determined that the subject is homozygous in the mutated NQO1 gene.

17. A method as recited in claim 16, wherein the inhibitor of the apoptotic protein is selected from a group comprising Bad and Bid.

18. A method comprising:
selecting a subject that has above normal risk of Painful Bladder Syndrome (PBS) or is expressing a symptom of PBS; and
treating the subject with a therapeutically effective amount of an antioxidant.

19. A method as recited in claim 18, further comprising:
obtaining a biological sample from the subject;
analyzing the biological sample to determine whether the subject is homozygous in a mutated NQO1 gene;
administering a different second treatment if the subject is determined to be homozygous in the mutated NQO1 gene; and
administering a different third treatment if the subject is determined to be not homozygous in the mutated NQO1 gene.

20. A method as recited in claim 19, wherein:
analyzing the biological sample further comprises analyzing the biological sample to detect the presence of a mutated form of a NAD(P)H dehydrogenase [quinone] protein, abbreviated NQO1; and
determining whether the subject is homozygous in the mutated NQO1 gene further comprises determining that the subject is homozygous in the mutated NQO1 gene if the mutated form of NQO1 is detected in the biological sample and NQO1 is not detected in the biological sample.
